(19) United States
(12) Patent Application Publication
(10) Pub. No.: US 2011/0020359 A1
(43) Pub. Date: Jan. 27, 2011

(54) METHODS AND COMPOSITIONS FOR TREATING NEURONAL DAMAGE OR DEGENERATION

(75) Inventors: Linda C. Hsieh-Wilson, San Marino, CA (US); Joshua Micah Brown, Pasadena, CA (US); Binquan Zhuang, Pasadena, CA (US)

Correspondence Address: WILSON, SONSINI, GOODRICH & ROSATI, 650 PAGE MILL ROAD, PALO ALTO, CA, 94304-1050, US

(73) Assignee: California Institute of Technology, Pasadena, CA (US)

(21) Appl. No.: 12/843,758

(22) Filed: Jul. 26, 2010

Related U.S. Application Data
(60) Provisional application No. 61/271,705, filed on Jul. 24, 2009.

(51) Int. Cl. A61K 39/395; A61K 38/16; A61P 25/00

(52) U.S. Cl. 424/141.1; 424/130.1; 514/21.2

(57) ABSTRACT

This invention provides methods and compositions for reducing chondroitin-sulfate-proteoglycan-mediated inhibition of neuronal growth. The methods and compositions provided herein are particularly useful for treatment of neuronal damage or degeneration.

### RELATED APPLICATIONS

This application claims the benefit of U.S. Provisional Application No. 61/271,705, filed Jul. 24, 2009, which is incorporated by reference herein in its entirety.

### STATEMENT AS TO FEDERALLY SPONSORED RESEARCH

This invention was made with government support under R01GM093627-01 awarded by the National Institutes of Health. The government has certain rights in the invention.

### BACKGROUND OF THE INVENTION

Each year more than 20 million people suffer from nerve damage or degeneration. Nerve damage or degeneration can be caused by a number of factors including various diseases, nutritional deficiencies, mechanical impact or trauma, and adverse effects of existing drugs. The common diseases that can manifest in nerve damage or degeneration are various types of motor neuron disorders, diabetic neuropathy, infectious diseases, neoplastic conditions, and autoimmune diseases. Nerve damage is most commonly observed in patients suffering from autoimmune diseases such as multiple sclerosis, Guillain Barre syndrome, myasthenia gravis, lupus, and even inflammatory bowel disease. Certain infectious diseases can also cause nerve damage or degeneration. These infectious diseases include Lyme disease, herpes virus, HIV, and hepatitis C. A wide variety of mechanical impact and trauma can cause varying degrees of nerve damages including pinched or severed nerves, spinal cord injuries, and autonomic, sensory, or motor nerve injuries. Additionally, neurodegenerative diseases including but not limited to Parkinson's (degeneration of the dopaminergic nigrastriatal pathway), Alzheimer's (degeneration of neurons in the cerebral cortex and certain subcortical regions), and Huntington's disease (degeneration of striatal neurons) can cause or contribute to nerve degeneration.

Current treatments for nerve damage or degeneration are primarily palliative and not curative. In particular, there is no effective treatment for regenerating or repairing damaged nerves. One major obstacle to axonal regeneration is the growth-inhibitory extracellular environment of the encountered axons. This inhibitory activity is principally attributable to components of CNS myelin and molecules present in the extracellular and cell-surface environment. These components include a variety of proteins, proteoglycans or polysaccharides, of which glycosaminoglycans are thought to play a key role in mediating these inhibitory signals.

Glycosaminoglycans have an inherent capacity to encode functional information that rivals DNA, RNA and proteins. Specifically, these polysaccharides display diverse patterns of sulfation that are tightly regulated in vivo Kitagawa, H. et al., J. Biol. Chem. 272, 31377-31381 (1997) and Plaas, A. H. K. et al., J. Biol. Chem. 273, 12642-12649 (1998). Chondroitin sulfate proteoglycans (CSPGs) such as neurocan, phosphacan, NG2 and brevican are upregulated following CNS injuries and have been implicated as potent inhibitors of axonal regeneration. CSPGs are a class of glycosaminoglycans composed of chondroitin sulfate (CS) polysaccharides consisting of units of the repeating disaccharide D-glucuronic acid (GlcA) and N-acetyl-D-galactosamine (GalNAc). The sugar hydroxyls may be variably sulfated to give rise to diverse sulfation patterns. Chemical structures of major sulfation motifs found in the mammalian nervous system include CS-A (GlcA-4SGalNAc), CS-C (GlcA-6SGalNAc) and CS-E (GlcA-4S, 6SGalNAc). CSPGs are thought to interact with a variety of proteins and other signaling molecules including but not limited to the cell surface protein p75^{NTR}, as well as other extracellular proteins. These cell surface receptors and extracellular proteins in turn may transduce binding events into an inhibitory signal via downstream intracellular mediators of CS signaling.

### SUMMARY OF THE INVENTION

The present invention addresses the need for alternative treatments for neuronal damage and other neurodegenerative diseases existing in the art.

In one embodiment, the present invention provides a method for promoting neuronal regeneration in a subject in need thereof. The method comprises administering to said subject a pharmaceutical composition comprising a physiologically acceptable carrier and an agent that inhibits chondroitin sulfate E (CS-E) mediated signaling. The agent used in the subject method is typically present in an amount effective in promoting neurite outgrowth in said subject. In one aspect of the embodiment, the pharmaceutical composition administered to said subject comprises a chondroitin sulfate E (CS-E) binding agent. In another aspect, the method comprises inhibiting CS-E mediated signaling, wherein said signaling comprises intracellular signaling mediated by one or more cellular proteins selected from signaling molecules including but not limited to mitogen activated protein kinase kinase (MEK), epidermal growth factor receptor (EGFR), protein kinase C (PKC), rho kinase (RhoK), and RhoA.

In another embodiment, the present invention provides a method of treating a subject suffering from a neural injury comprising administering to said subject in need thereof a pharmaceutical composition comprising therapeutically effective amount of a chondroitin sulfate E (CS-E) binding agent and a physiologically acceptable carrier.

In another embodiment, the present invention provides a method of reducing chondroitin proteoglycan (CSPG)-mediated inhibition of neuronal growth. The method typically comprises: contacting a biological sample comprising one or more extracellular matrix components with a chondroitin sulfate E (CS-E) binding agent under conditions sufficient to reduce CS-E mediated signaling in said neuronal cell.

In a further aspect of any one of the foregoing embodiments, the methods of the present invention provide for administering chondroitin sulfate E (CS-E) binding agents that are antibodies that selectively bind to CS-E. The antibodies may be polyclonal or monoclonal, or a fragment thereof.

In a further aspect of any one of the foregoing embodiments, the methods of the present invention provide for administering chondroitin sulfate E (CS-E) binding agents that are soluble proteins and can selectively bind CS-E. The soluble proteins are comprised of soluble domains of one or more of p75^{NTR} NgR, Nogo, OMgp, Ephrin receptor B3, Ephrin A3, Ephrin receptor A4, Ephrin B1, which selectively bind to CS-E.

In a further aspect of any one of the foregoing embodiments, the methods of the present invention provide for administering a chondroitin sulfate E (CS-E) binding agent that inhibits CS-E signaling. Inhibition of CS-E signaling is evidenced by, e.g., reduction of RhoA activation by CS-E in a neuronal cell or any cell expressing p75^{NTR} or reduction of binding of CS-E to a surface of a neuronal cell or a cell expressing p75^{NTR}.

In a further aspect of any one of the foregoing embodiments, the methods of the present invention provide for administering a chondroitin sulfate E (CS-E) binding agent that inhibits CS-E mediated growth cone collapse.

In a further aspect of any one of the foregoing embodiments, the methods of the present invention provide for administering a chondroitin sulfate E (CS-E) binding agent that promotes neurite outgrowth in vitro.

In a further aspect, the methods of the present invention provide for administering a chondroitin sulfate E (CS-E) binding agent that exhibits a strong binding affinity for CS-E (e.g., a Kd value less than about 100 nM, 200 nM, 300 nM, 400 nM, 500 nM, 600 nM, 700 nM, 800 nM, 900 nM, 1 μM, or 1 mM) as ascertained in an in vitro binding assay. The binding assay may be a surface plasmon resonance assay, such as in Example 9.

In yet another aspect, the present invention provides for administering a chondroitin sulfate E (CS-E) binding agent that promotes or improves locomotor function, grasping, bladder function, neuromuscular functional recovery, or walking in a subject. The subject may be suffering from spinal cord injury, optical nerve injury, motor nerve injury, stroke, a neurodegenerative disease, or a combination thereof.

In yet another embodiment, the present invention provides a pharmaceutical composition comprising a chondroitin sulfate E (CS-E) binding agent and a physiologically acceptable carrier. In one aspect of this embodiment, the chondroitin sulfate E (CS-E) binding agent is an antibody exhibiting binding selectively to CS-E. In another aspect of this embodiment, the chondroitin sulfate E (CS-E) binding agent is a monoclonal antibody exhibiting binding selectively to CS-E. In another aspect, the chondroitin sulfate E (CS-E) binding agent is a soluble protein exhibiting binding selectively to CS-E. In yet another aspect, the chondroitin sulfate E (CS-E) binding agent is a soluble protein exhibiting binding selectively to CS-E, wherein the soluble protein comprises a domain of NgR that selectively binds to CS-E. In a further aspect, the chondroitin sulfate E (CS-E) binding agent is a soluble protein exhibiting binding selectively to CS-E, wherein the soluble protein comprises a domain of p75^{NTR} that selectively binds to CS-E. In still another aspect, the chondroitin sulfate E (CS-E) binding agent is a soluble protein exhibiting binding selectively to CS-E, wherein the soluble protein comprises a domain of Nogo, OMgp, or Ephrin receptor B3 (EphB3), Ephrin A3 (Efn A3), Ephrin receptor A4 (EphA4), Ephrin B1 (Efn B1) that selectively binds to CS-E. In yet another aspect, the chondroitin sulfate E (CS-E) binding agent inhibits CS-E signaling as evidenced by inhibition of RhoA activation in a neuronal cell or a cell expressing p75^{NTR} or inhibition of binding of CS-E to a surface of a neuronal cell or a cell expressing p75^{NTR}. In yet still another aspect, the chondroitin sulfate E (CS-E) binding agent promotes neurite outgrowth in vitro, or improves locomotor function, grasping, bladder function, neuromuscular functional recovery, or walking in a subject.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: depicts CS polysaccharides composed of units of the repeating disaccharide D-glucuronic acid (GlcA) and N-acetyl-D-galactosamine (GalNAc). The sugar hydroxyls are variably sulfated to give rise to diverse sulfation patterns. Chemical structures of major sulfation motifs found in the mammalian nervous system: CS-A (GlcA-4SGalNAc), CS-C (GlcA-6SGalNAc) and CS-E (GlcA-4S, 6SGalNAc).

FIG. 2: shows that the CS-E sulfation motif inhibits DRG neurite outgrowth and induces growth cone collapse. (A) Dissociated chick E7 DRGs are cultured on a substratum of poly-DL-ornithine (P-Orn control), chicken brain-derived CSPGs, chondroitinase ABC-treated CSPGs, or CS polysaccharides enriched in the CS-A, CS-C or CS-E sulfation motifs. Representative images and a bar graph measuring average neurite length (±SEM, error bars) from three experiments (n=50-200 cells per experiment) are shown. Scale bars, 100 μm. (B) Polysaccharides enriched in the CS-E sulfation motif, but not the CS-A or CS-C motifs, inhibit DRG neurite outgrowth in a dose-dependent manner. (C) CS-E-enriched polysaccharides repel axon crossing in a boundary assay. Polysaccharides (1 mg/ml) or PBS control are mixed with Texas Red and spotted on P-Orn coated coverslips. Dissociated rat P5-9 CGN neurons are immunostained with an anti-βIII-tubulin antibody. Representative images and a bar graph measuring percentage of axon crossing (±SEM, error bars) from two experiments (n=30-50 axons per experiment) are shown. Scale bars, 50 μm. (D) CS-E-enriched polysaccharides, but not CS-A- or CS-C-enriched polysaccharides, induce growth cone collapse. DRG explants from chick E7-9 embryos are grown on a P-Orn/laminin substratum, treated with medium (control) or the indicated polysaccharides, and stained with rhodamine-phalloidin. Representative images and a graph measuring percentage of growth cone collapse (±SEM, error bars) from five experiments (n=50-100 growth cones per experiment) are shown. Arrows indicate collapsed growth cones. (E) Structures of synthetic glycopolymers displaying pure CS-A, CS-C and CS-E disaccharides. (F) The synthetic CS-E glycopolymer inhibits DRG neurite outgrowth, whereas glycopolymers containing pure CS-A or CS-C sulfation motifs have little effect. (G) The synthetic CS-E glycopolymer, but not the CS-A or CS-C glycopolymer, induces DRG growth cone collapse. All statistical analyses were performed using the one-way ANOVA (*P<0.0001, relative to control).

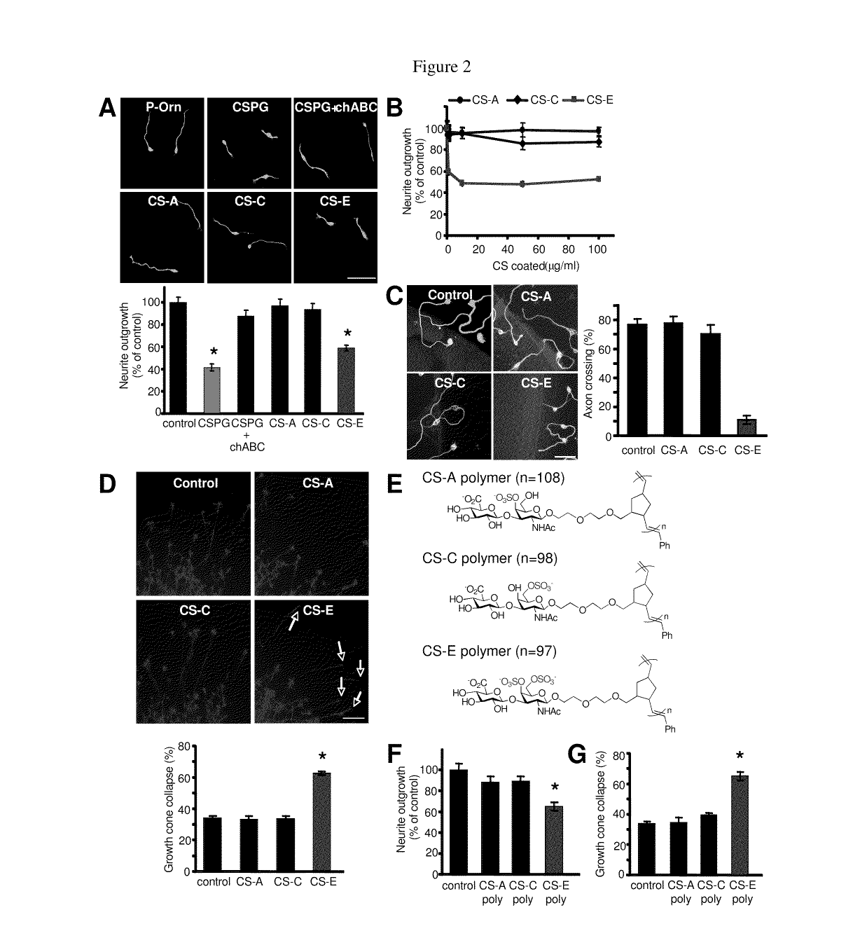

FIG. 3: illustrates that the CS-E sulfation motif is found on CSPGs and activates ROCK, EGFR, and MAP kinase pathways. (A) Anti-CS-E monoclonal antibody binds selectively to CS-E-enriched polysaccharides on carbohydrate microarrays. Little binding to other sulfated CS polysaccharides and glycosaminoglycan classes is detected. Microarrays are incubated with anti-CS-E antibody, followed by a Cy3-conjugated anti-mouse IgG secondary antibody, and analyzed using a GenePix 5000a scanner. Experiments are done in triplicate (n=10 per condition). (B) Dose-dependent binding of the anti-CS-E antibody to CSPGs, as shown in an enzyme-linked immunosorbent assay binding curve. Average values (±SD, error bars) are shown for one representative experiment. The experiment is repeated three times with similar results. (C) Both CS-E polysaccharides and CSPGs require activation of MEK, EGFR, and ROCK for inhibition of neurite outgrowth. Inhibitors against MEK (PD98059, 25 EGFR (AG1478, 15 nM), and ROCK (Y27632, 5 μM) rescue CS-E- and CSPG-mediated inhibition of neurite outgrowth in dissociated rat P5-9 CGN cultures, whereas JNK inhibitor II (10 μM) has no effect. Quantification from at least three experiments is reported. Treatment with each inhibitor alone has no effect on neurite outgrowth when compared to untreated neuronal cultures (fig. S8). (One-way ANOVA, *P<0.0001, relative to CS-E control without inhibitors, **P<0.0001, relative to CSPG control without inhibitors; n=50-200 cells per experiment).

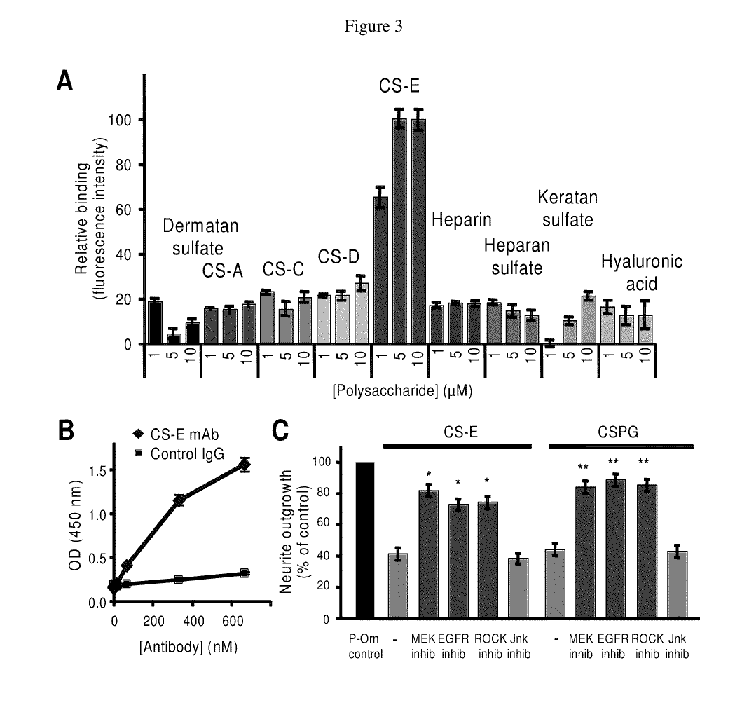

FIG. 4: illustrates that CS-E interacts with NgR and may require p75^{NTR} to inhibit neurite outgrowth. (A) NgR binds selectively to CS-E-enriched polysaccharides on carbohydrate microarrays. Microarrays are incubated with NgR-Fc, followed by a Cy3-conjugated anti-mouse IgG secondary antibody, and analyzed using a GenePix 5000a scanner. Graphs show quantification from six experiments (n=10 per condition). (B) Pull-down of full-length, native NgR by CS-E. COS-7 cell lysates expressing full-length myc-NgR1 and HA-p75^{NTR} are incubated with biotinylated CS-E-enriched polysaccharides bound to streptavidin beads. NgR binding is detected by immunoblotting with an anti-myc antibody. Similar results are obtained using COS-7 lysates expressing myc-NgR1 alone, and no binding to HA-p75^{NTR} is detected. The experiment is repeated in triplicate. (C) Neurite outgrowth inhibition mediated by CS-E and CSPGs is significantly attenuated in p75^{NTR}-deficient neurons. CGN neurons from wild type or p75^{NTR}-deficient mice are cultured on a substratum of P-Orn and CS-E or CSPGs. Neurites are visualized by staining with an anti-βIII-tubulin antibody, and the percent neurite outgrowth is quantified relative to the wild-type control. Quantification from three experiments is reported. (One-way ANOVA, *P<0.0001, relative to wild-type P-Orn control, **P<0.0001, relative to p′75−/− P-Orn control; n=50-200 cells per experiment).

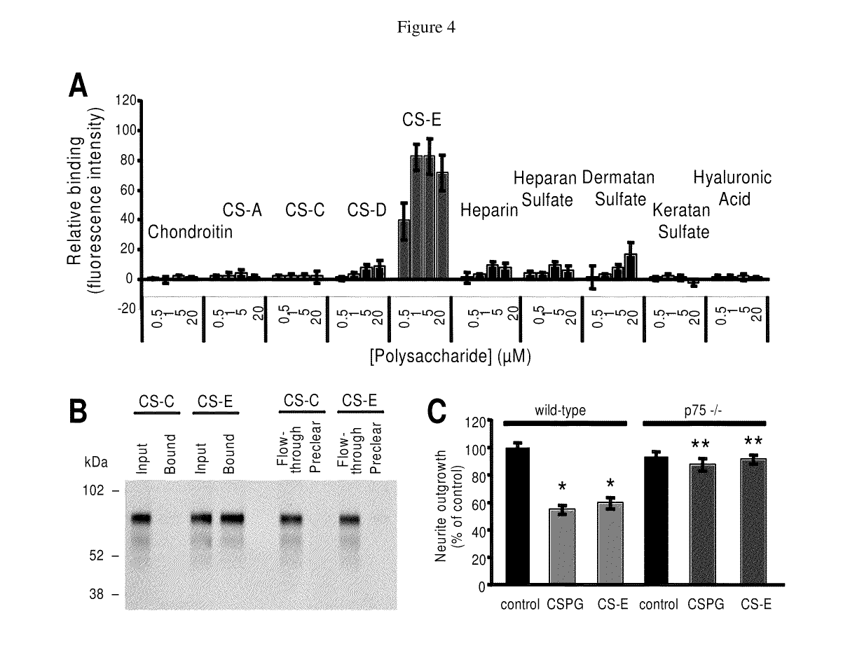

FIG. 5: illustrates that the CS-E-specific antibody blocks CSPG-mediated growth inhibition and promotes optic nerve regeneration in vivo. (A) Anti-CS-E antibody, but not anti-CS-A or IgG control antibody, reverses inhibition of neurite outgrowth mediated by CSPGs. Dissociated chick E7 DRGs are cultured on a substratum of P-Orn (control; black) or CSPGs (0.5 μg/ml; grey or blue) in the presence of the indicated antibodies (0.1 mg/ml) for 12 hr. Treatment with each antibody alone (black bars) has no effect on neurite outgrowth relative to untreated neurons cultured on P-Orn. Neurites are visualized by staining with an anti-βIII-tubulin antibody, and quantification from three experiments is shown (One-way ANOVA, *P<0.0001, relative to CSPG without antibody treatment control; n=50-200 cells per experiment). (B) (a, b) Induction of CS-E expression after optic nerve injury. Immunofluorescence labeling of CS-E expression in optic nerve sections at day 1 after sham-operation (a) or optic nerve crush injury (b). Note upregulation of CS-E within a 200-500 μm radius around the injury site (marked by an asterisk). (c to f) Anti-CS-E antibody promotes optic nerve regeneration in vivo. Representative epifluorescence photomicrographs of optic nerve sections taken from mice treated with control IgG (c), anti-CS-E antibodies (d), chondroitinase ABC (e) or chondroitinase ABC plus anti-CS-E antibody (f). Retinal ganglion cell axons (red) are labeled by an anterograde axon tracer, CTB, which is injected into the vitreous 3 days prior to sacrifice, followed by immunostaining with goat anti-CTB antibody. (g to i) High magnification images of insets in c, d, and e, respectively. Scale bars, 75 μm (a-f); 25 μm (g-i). Arrowheads indicate growth cone structures. (C) Quantification of axon regeneration in vivo. Nerve fibers are counted at 125-μm intervals from the crush site from three nonconsecutive sections, and the number of fibers at a given distance is calculated (±SEM, error bars). Both the anti-CS-E- and chABC-treated groups show significantly more regenerating axons as compared with the control IgG antibody-treated group (ANOVA with Bonferroni posttests at each distance, *P<0.001 as compared to controls; n=6 for each group). (D) Quantification of the distances of axon regeneration. Longest distance of axon regeneration is measured from at least 4 nonconsecutive optic nerve sections from each mouse (±SEM, error bars). Combined treatment of CS-E mAb and 8-(4-chlorophenylthio)-cyclic AMP (CPT-cAMP) more than doubles the distance of axon regeneration but does not affect the number of regenerating axons compared to the anti-CS-E or CPT-cAMP treatment alone.

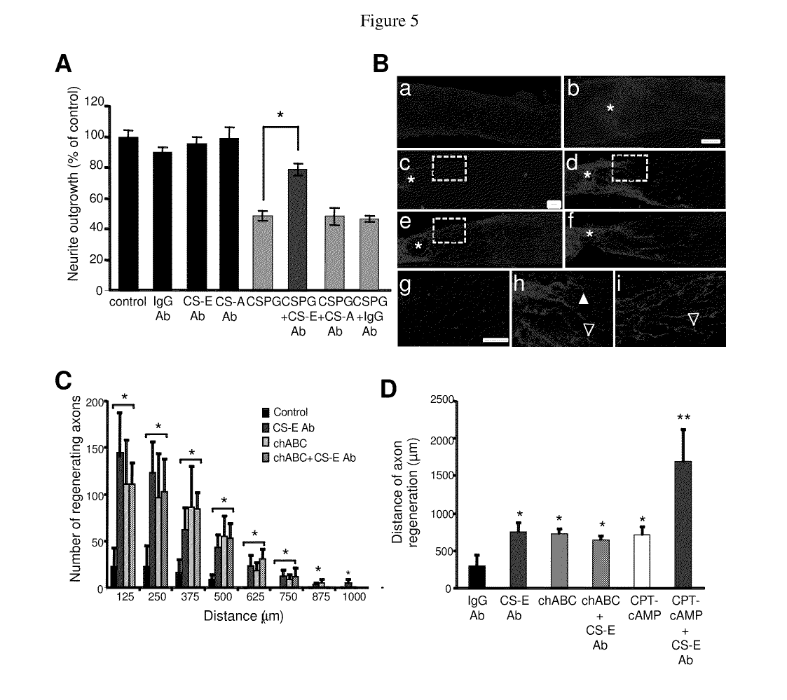

FIG. 6: illustrates that polysaccharides enriched in the CS-E sulfation motif (˜60% CS-E content), but not CS-A or CS-C, inhibit the neurite outgrowth of cerebellar granule neurons (CGNs). (A) Dissociated P5-9 rat CGNs are cultured on a substratum of polysaccharides enriched in the CS-A, CS-C or CS-E sulfation motifs (1 μg/ml) for 24 h. Cells are immunostained using an anti-βIII-tubulin antibody, imaged and quantified using the NIH software Image J. Representative images are shown on the top, and a graph measuring the average neurite length (±SEM, error bars) from at least three experiments is shown on the bottom (One-way ANOVA, *P<0.0001, relative to P-Orn control; n=50-200 cells per experiment). (B) Polysaccharides enriched in the CS-E sulfation motif, but not the CS-A or CS-C motifs, inhibit CGN outgrowth in a dose-dependent manner.

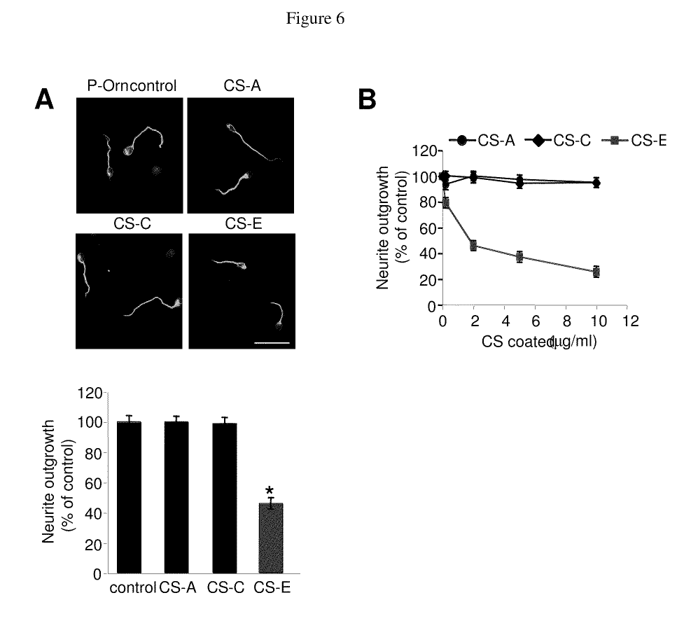

FIG. 7: depicts representative images of the (A) axon repellant activity of CS-A and CS-E polysaccharides at high sugar concentrations (10 mg/ml), (B) inhibition of chick E7 DRG outgrowth by the synthetic glycopolymers, and (C) growth cone collapse of chick E7-9 DRG explants induced by the synthetic glycopolymers. Arrows indicate collapsed growth cones. Scale bars, 100 μm.

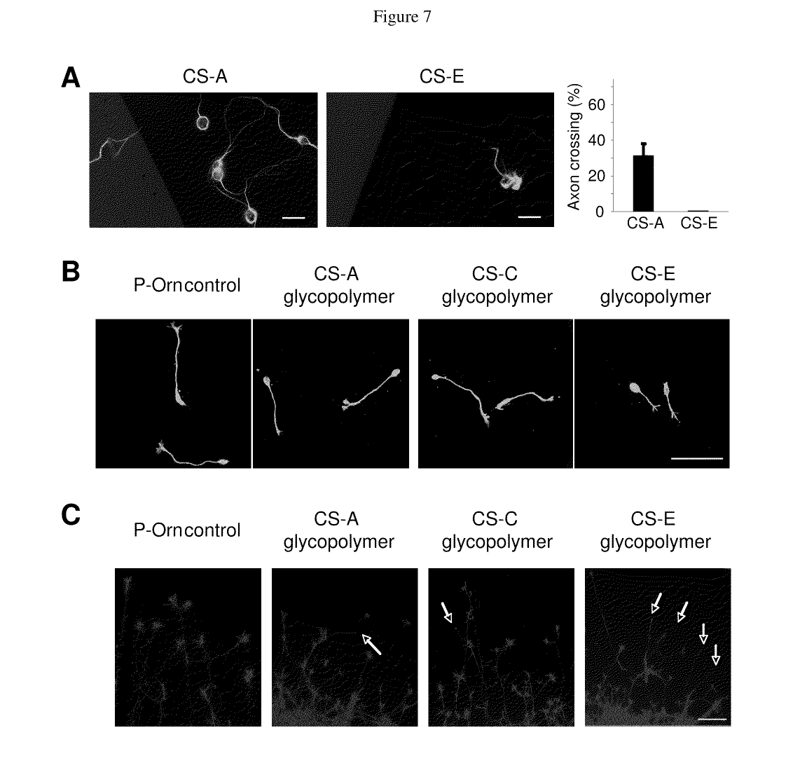

FIG. 8: illustrates that the anti-CS-E antibody binds selectively to a pure, synthetic CS-E tetrasaccharide and CS-E-enriched polysaccharides, whereas it does not bind to CS-A or CS-C tetrasaccharides or polysaccharides. (A) Tetrasaccharides containing pure CS-A, CS-C or CS-E motifs are conjugated to bovine serum albumin (BSA) and spotted on nitrocellulose membranes at the indicated amounts. Binding of the antibody to the membrane is detected using a goat anti-mouse secondary antibody conjugated to Alexa Fluor® 680 and imaged using an Odyssey Infrared Imaging System. The anti-CS-E antibody binds in a concentration-dependent manner to the BSA-CS-E tetrasaccharide conjugate but does not bind significantly to BSA-CS-A, BSA-CS-C, or BSA alone. (B) Binding of the anti-CS-E antibody to biotinylated CS polysaccharides enriched in the CS-A, CS-C, or CS-E sulfation motifs. Biotinylated CS polysaccharides are adsorbed on streptavidin-coated plates, and antibody binding to the plate is detected using a goat anti-mouse secondary antibody conjugated to horseradish peroxidase. Experiments are repeated in triplicate.

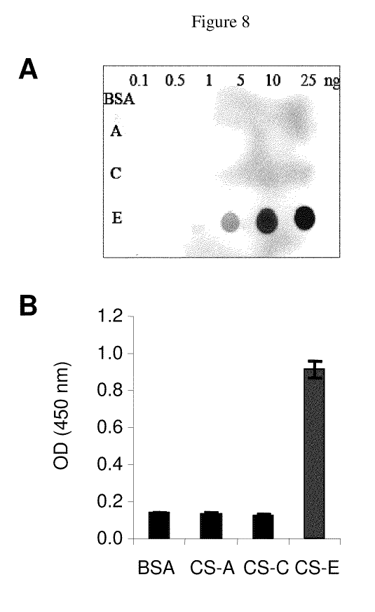

FIG. 9: depicts the results of kinetic analysis of the interaction between the anti-CS-E antibody and CS-E tetrasaccharide by surface plasmon resonance. (A) The synthetic CS-E tetrasaccharide is covalently immobilized onto the surface via reductive amination chemistry. Binding kinetics are monitored at 25° C. by injecting the CS-E antibody over the surface for 300 s at 30 μL·min⁻¹ and recording the dissociation for 900 s. The surface is regenerated with 6 M guanidine HC1. The resulting sensorgrams are fit to the bivalent analyte model. According to this model, a surface-bound analyte can bind another ligand molecule with the free binding site. The kinetic parameters of the fit, with standard errors in parenthesis, are tabulated in (C). (B) The affinity is also measured by injecting the antibody over the surface for 3600 s to give sufficient time to reach equilibrium. The response at equilibrium is plotted versus concentration to give a K_{D} value of 4.3 nM.

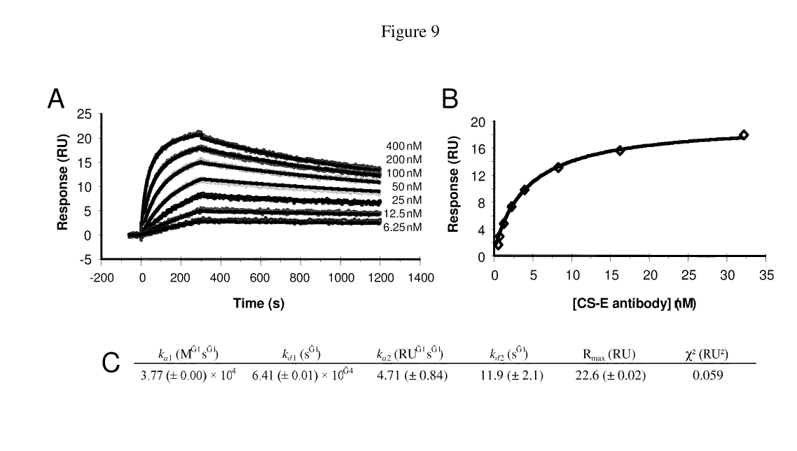

FIG. 10: illustrates that the Fc domain and the axon guidance receptor EphB2 do not bind to CS-E polysaccharides on carbohydrate microarrays. 10(A) depicts the binding of NgR-Fc to the array. 10(B) depicts the control Fc, which does not exhibit binding to the arrays. (C) EphB2-Fc does not bind to glycosaminoglycans on carbohydrate microarrays. Each bar represents an average of 10 spots from one typical array. Relative binding is plotted by comparison to the fluorescence intensity of NgR-Fc binding to 5 μM CS-E.

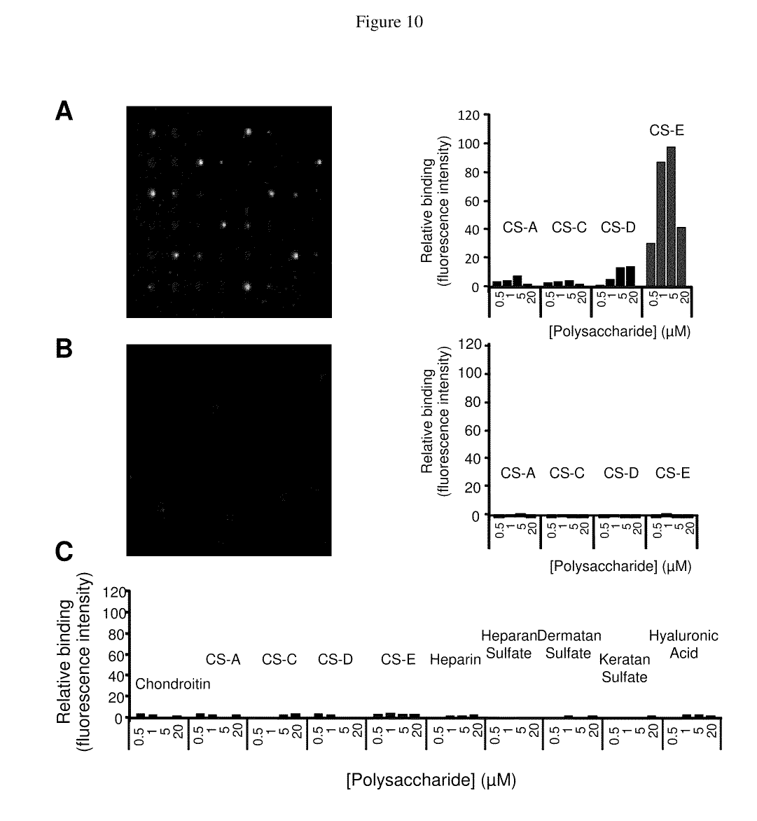

FIG. 11: depicts the results of kinetic analysis of NgR-binding to CS-E-enriched polysaccharides by surface plasmon resonance. (A) Biotinylated CS-E polysaccharides are immobilized onto the surface via streptavidin capture (R_{L}=25 RU). NgR is passed over the surface at 80 μL·min⁻¹ at 25° C. for 240 s. After monitoring the dissociation for 600 s, the surface is regenerated by three successive injections of 2.5 M MgCl₂ of 90 s at 30 μL·min⁻¹. The resulting sensorgrams are fit to the heterogeneous ligand model (black lines), where the bulk refractive index (RI) is set as a constant value of zero. The model predicts two independent binding sites. The kinetic parameters derived from the fitting are tabulated in (C), with the standard error in parentheses. (B) Simulation of curves representing the individual binding components of the heterogenous ligand model. A binding site with picomolar affinity (solid lines) binds approximately 6 molecules of NgR per polysaccharide, based on solving the equation Rₘₐₓ=R_{L}·(MW_{A}/MW_{L})·Sₘ for Sₘ, the number of binding sites. The observed nanomolar binding site (dashed lines) binds approximately 2 molecules of NgR per polysaccharide. (D) No binding of NgR to CS-C polysaccharides was observed (black line).

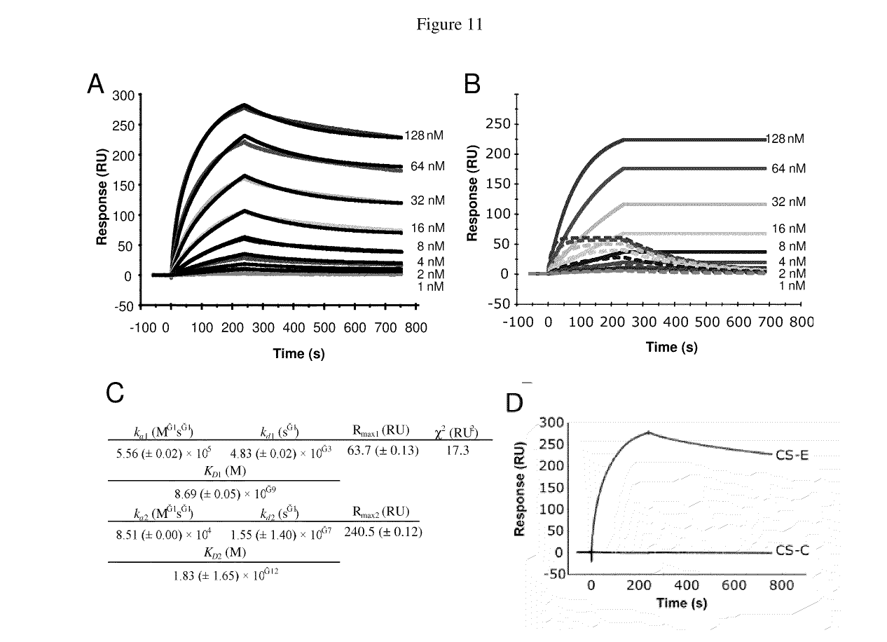

FIG. 12: depicts the results of binding specificity of tested molecules to CS-E containing microarray. (A) EphB3-Fc binds to CS-E with high specificity by microarray analysis. (B) No binding to CS-E, nor any other glycosaminoglycan tested in A was detected for EphB2-Fc (data not shown). (C) Surface plasmon resonance of EphB3-Fc binding to CS-E polysaccharide. Data show binding at the following concentrations: 3072, 1536, 768, 384, 192, 96, 48, 24, 12, 6, 3 nM. (D) A comparison of EphB3-Fc and EphB2-Fc binding to CS-E by SPR. While high concentrations of EphB3 gives a strong response by SPR (black, 3 μM; red, 1.5 μM), EphB2 shows no binding to CS-E at these concentrations. (E) Tabulation of the binding rate constants of the EphB3-Fc/CS-E interaction. (F) Full-length EphB3 is pulled down by CS-E, but not CS-C, as shown on an immunoblot.

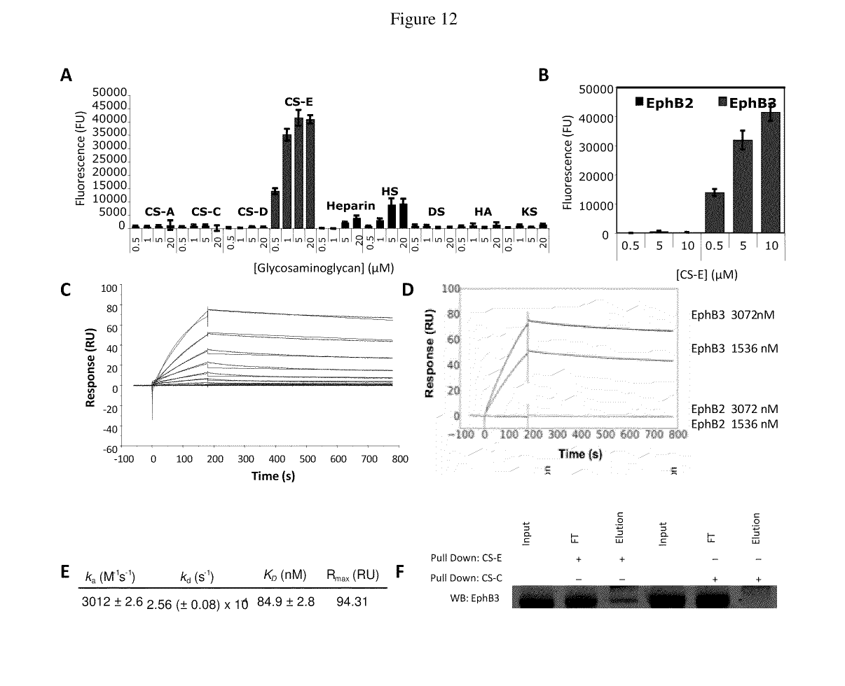

FIG. 13: illustrates that under the conditions tested, an anti-CS-E antibody does not affect the survival or intrinsic growth status of retinal ganglion cells. (A) Application of anti-CS-E antibody does not change retinal ganglion cell survival after optic nerve injury. Bar graph indicates relative survival of retinal ganglion cells in control IgG or anti-CS-E antibody-treated mice that are quantified at 14 days post optic nerve injury. (B) Comparison of axon regeneration in vivo induced by anti-CS-E and/or CPT-cAMP. Retinal ganglion cell axons are counted at 125-μm intervals from the crush site from three nonconsecutive sections, and the number of fibers at a given distance is calculated (±SEM, error bars). ANOVA with Bonferroni posttests at each distance, *P<0.001 as compared to controls; n=6 for each group.

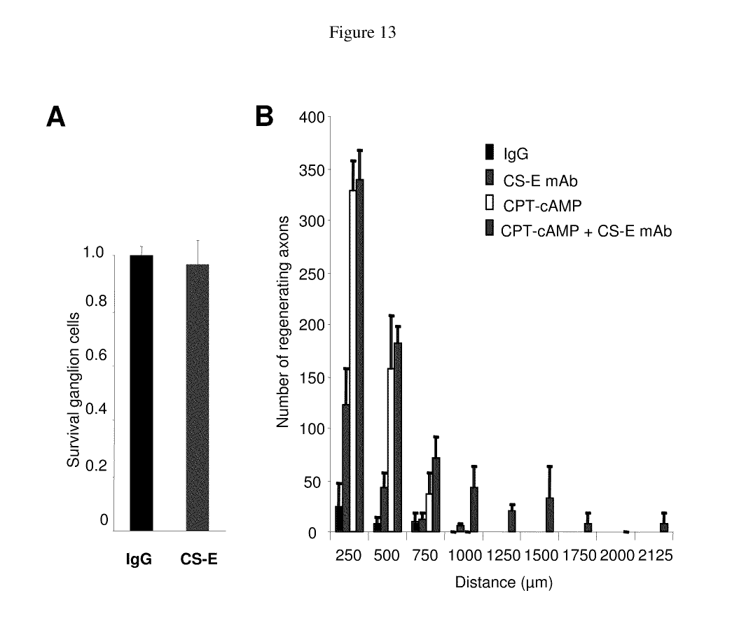

FIG. 14: illustrates data from an oligosaccharide microarray experiment for detection and analysis of chondroitin sulfate binding agents. The results demonstrate that under the conditions tested, Nogo-66 (top panel), the amino terminal domain of Nogo (middle panel), and oligodendrocyte myelin protein (OMgp) (bottom panel) bind to CS-E polysaccharides. Nogo and OMgp have been implicated in inhibiting neuronal growth after injury.

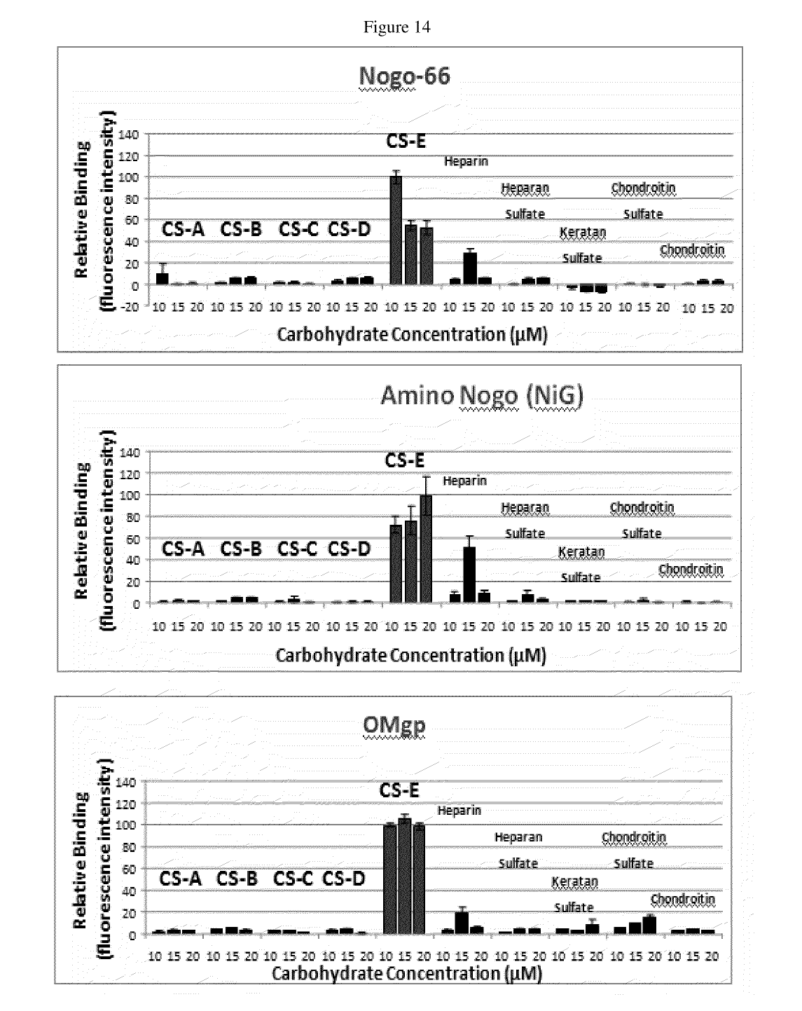

FIG. 15: illustrates data from carbohydrate microarray and surface plasmon resonance analyses for the binding of Ephrin receptors to CS-E polysaccharides and other glycosaminoglycans. The relative binding of proteins EphA1, EphA2, EphA3, EphA4, EphA5, EphA6, EphA7 and EphA8 are examined for interactions with Chondroitin, CS-A, CS-C, CS-D, CS-E, Dermatan Sulfate, Heparan Sulfate, Heparan, and Hyaluronic Acid at varying polysaccharide concentrations of 0.5, 1, 5, and 20 μM. Results demonstrate variance among Ephrin receptor species for specific binding to CS-E-enriched polysaccharides.

FIG. 16: illustrates data from carbohydrate microarray and surface plasmon resonance analyses for the binding of ephrins, ligands that activate Ephrin receptors, to CS-E polysaccharides and other glycosaminoglycans. The relative binding of proteins Ephrin A1, Ephrin A2, Ephrin A3, Ephrin A4, Ephrin A5, Ephrin B1, Ephrin B2, and Ephrin B3 are examined for interactions with Chondroitin, CS-A, CS-C, CS-D, CS-E, Dermatan Sulfate, Heparan Sulfate, Heparan, and Hyaluronic Acid at varying polysaccharide concentrations of 0.5, 1, 5, and 20 μM. Results demonstrate variance among Ephrin species for specific binding to CS-E-enriched polysaccharides.

FIG. 17: illustrates data from EphA4 knockout mouse assays. The results demonstrate that under the conditions tested, CGN neurons from EphA4-deficient mice show reduced inhibition of neurite outgrowth by CS-E as compared to control mice.

FIG. 18: illustrates data from competition binding assays. Results demonstrate that CS-E antibody can compete for binding of Nogo Receptor (NgR) to CS-E polysaccharides on carbohydrate arrays. In addition, it is also shown that CS-E antibody blocks the inhibitory effects of CS-E polysaccharides on rat cerebellar granule neurons. In FIG. 18A, the CS-E antibody blocks binding of NgR-Fc to CS-E on the array. The binding reagents mixed are: NgR alone (200 nM, left), NgR and CSE- Ab (200 nM and 10 μM, respectively, middle) or CS-E Ab alone (10 μM, right). In FIG. 18B, the inhibitory effect of CS-E polysaccharides (CS-E poly) is blocked by the CS-E antibody in CGNs. P7 rat CGNs are grown for 24 hours on CS-E (1 μg/ml) in the presence of the CS-E antibody (CS-E Ab; 10 μg/ml). Digestion of CS-E polysaccharides with chondroitinaseABC (CHase) results in a small extent of residual inhibition, which is completely blocked by the CS-E antibody.

FIG. 19: illustrates immunostaining of lesioned rat cortex and spinal cord with the anti-CS-E antibody. Specifically, sections from rat brains with cortical lesions at 1 day, 3 days, and 7 days post lesion were immunostained with the CS-E antibody. Increased immunostaining around the lesion at all three time points was recorded. Levels of staining appeared highest at 3 days post-lesion. Immunostaining was also assessed 2 days following a dorsal funiculus lesion of the rat spinal cord. As with cortical lesions, there was a general increase in immunostaining in the lesion margin, and some cells around the lesion expressed CS-E at a fairly high level. Some cells in the gray matter also stained for CS-E, but not as intensely as those near the lesion. Dorsal and ventral white matter also stained for CS-E in a pattern that resembled neurofilament expression. These results demonstrate that the CS-E sulfation motif is upregulated shortly after brain or spinal cord injury in vivo. These findings also suggest that the time course for treating with the CS-E antibody should likely be early, given the rapid increase in CS-E expression within 1-2 days post injury.

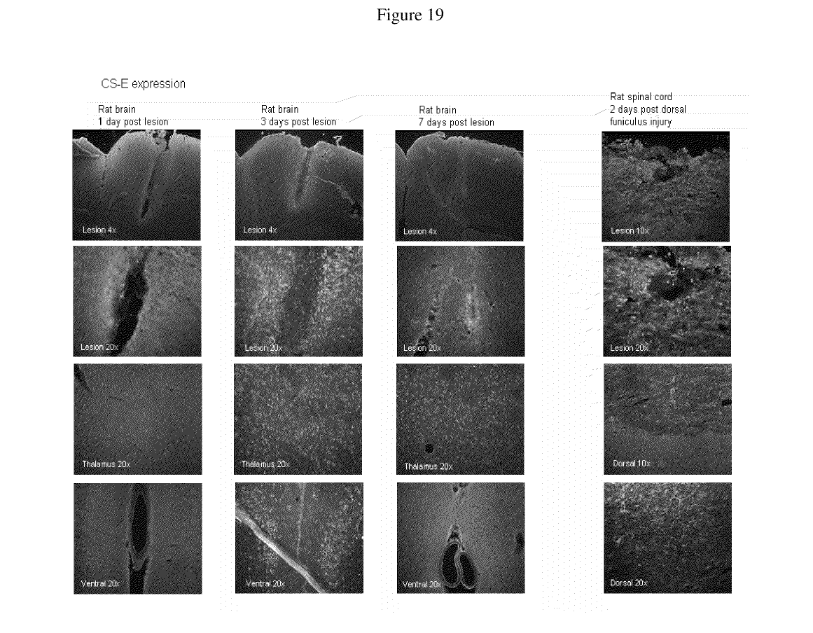

### DETAILED DESCRIPTION OF THE INVENTION

### I. Definitions

Unless defined otherwise, all technical and scientific terms used herein have the same meaning as is commonly understood by one of skill in the art to which this invention belongs.

As used in the specification and claims, the singular form “a”, “an” and “the” include plural references unless the context clearly dictates otherwise.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. axon growth. The specific dose will vary depending on the particular compounds chosen, the dosing regimen to be followed, whether it is administered in combination with other compounds, timing of administration, the tissue to which it is administered, or the physical delivery system in which it is carried.

As used herein, “treatment” or “treating,” or “palliating” or “ameliorating” is used interchangeably herein. These terms refer to an approach for obtaining beneficial or desired results including but not limited to therapeutic benefit and/or a prophylactic benefit. By therapeutic benefit is meant reduction, eradication or amelioration of the underlying disorder being treated. Also, a therapeutic benefit is achieved with the reduction eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “co-administration,” “administered in combination with,” and their grammatical equivalents, as used herein, encompass administration of two or more agents to an animal so that both agents and/or their metabolites are present in the animal at the same time. Co-administration includes simultaneous administration in separate compositions, administration at different times in separate compositions, or administration in a composition in which both agents are present.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist is associated with the development, or growth, of a neuron.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

As used herein, “agent”, “binding agent”, or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

“Subject” refers to an animal, such as a mammal, for example a human. The methods described herein can be useful in both human therapaeutics and veterinary applications. In some embodiments, the patient is a mammal, and in some embodiments, the patient is human.

The term “in vivo” refers to an event that takes place in a subject's body.

The term “in vitro” refers to an event that takes place outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

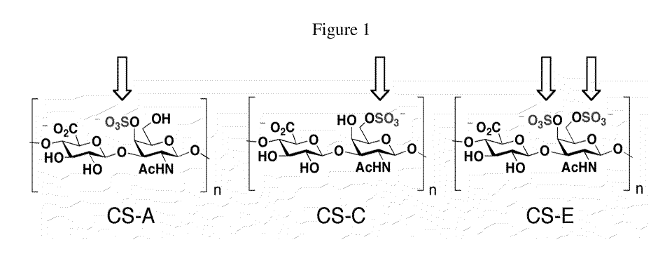

As used herein, the term “chondroitin sulfate” or “CS” refers to a molecule comprised of one or more repeating units of one or more of the chondroitin sulfate structures provided in FIG. 1. The term “chondroitin sulfate A” or “CS-A” refers to a molecule comprised of one or more repeating units of the CS-A structure provided in FIG. 1. Additionally, the term “chondroitin sulfate C” or “CS-C” refers to a molecule comprised of one or more repeating units of the CS-C structure provided in FIG. 1. And the term “chondroitin sulfate E” or “CS-E” refers to a molecule comprised of one or more repeating units of the CS-E structure provided in FIG. 1.

### II. Methods

The present invention provides methods for promoting neuronal regeneration in a subject. In one embodiment, the method includes the step of administering to said subject a pharmaceutical composition as provided herein comprising one or more compositions of the present invention that inhibit chondroitin sulfate (CS) mediated signaling, such as signaling via CS-A, CS-C, CS-E, or a combination thereof. An exemplary composition administered to a subject includes but is not limited to a composition comprising one or more binding agents that selectively bind CS-A, CS-C, CS-E, or a combination thereof. Other exemplary compositions to be used in the subject methods include but are not limited to agents that selectively bind to one or more of Nogo receptor (NgR), Nogo, Ephrin Receptor A4, Ephrin Receptor B3, Ephrin A3, p75^{NTR}, oligodendrocyte-myelin glycoprotein (OMgp) or a soluble fragment thereof as provided herein.

Additional compositions that are capable of inhibiting CS mediated signaling include but are not limited to NgR, Nogo, Ephrin receptor B3, p75^{NTR}, OMgp or a soluble fragment thereof, or a fusion or chimeric protein comprising a soluble fragment thereof. In still other cases, one can administer antagonists or inhibitors of downstream mediators of CS mediated signaling including inhibitors of MEK, epidermal growth factor receptor (EGFR), protein kinase C (PKC), RhoA, or other signaling molecules in the same or related pathways.

In yet other cases, siRNA or shRNA may be used to inhibit CS expression or downstream signaling events. For example, siRNA or shRNA may be used to silence expression of genes responsible for the biosynthesis or sulfation of CS (e.g., the GalNAc 4-sulfate 6-O-sulfotransferase that produces the CS-E sulfation pattern) or downstream mediators of CS signaling. In some cases, siRNA or shRNA may be used to silence expression of one or more of RhoA, MEK, NgR, Ephrin B3 receptor, or p75^{NTR} epidermal growth factor receptor (EGFR), protein kinase C (PKC) or other signaling molecules in the same or related pathways. Standard methods in the design of siRNA are known in the art (Elbashir et al., Methods 26:199-213 (2002)). In general, a suitable siRNA is between about 10-50, or about 20-25 nucleotides, or about 20-22 nucleotides. The target site typically has an AA dinucleotide at the 3′ end of the sequence, as siRNA with a UU overhang can be more effective in gene silencing. The remaining nucleotides generally exhibit homology to the nucleotides 3′ of the AA dinucleotides. In general, the siRNA typically exhibits at least about 50% homology to the target sequence, preferably at least about 70%, about 80%, 90% or even 95% homology to the target sequence. Where desired, potential target sites are also compared to the appropriate genome database, such that target sequences may have fewer than 70%, 60%, 50%, 40%, 30%, 20%, 10%, 5%, or even 1% homology to other genes. The readily available public database on the NCBI server, www.ncbi.nlm.nih.gov/BLAST is an example of a tool used to determine sequence homology. A public siRNA design tool is also readily available from the Whitehead Institute of Biomedical Research at MIT, jura.wi.mit.edu/pubint/http://iona.wi.mit.edu/siRNAext/.

The siRNA can also take the form of a hairpin siRNA (i.e. shRNA). One may vary a number of known factors in designing a suitable siRNA. Such variables include the selection of siRNA target sequence, the length of the inverted repeats that encode the stem of a putative hairpin, the order of the inverted repeats, the length and composition of the spacer sequence that encodes the loop of the hairpin, and the presence or absence of 5′-overhang can vary depending on the target and the predicted inverted region; all of which can be varied or customized according to standard procedures in the art. The stem can be 19 to 20 nucleotides long, preferably about 19, 21, 25, or 29 nucleotides long. The loop can range from 3 nucleotides to 23 nucleotides, with preference for loop sizes of about 3, 4, 5, 6, 7 and 9 nucleotides. Databases available to the public to aid in the selection and design of hairpin siRNA are also available, such as www.RNAinterference.org, and online design tools, for both hairpin siRNA and siRNA are available from commercial sites such as Promega and Ambion.

The methods provided herein are effective in inhibiting CS mediated signaling. Inhibition of CS mediated signaling such as CS-A mediated signaling, CS-C mediated signaling, or CS-E mediated signaling may be assayed by any methods known in the art as well as any methods provided herein. Non-limiting exemplary methods include assays for neuronal regeneration such as histochemical staining for neuronal specific markers as provided herein and further described in U.S. Patent Application Publication Nos. US20060177413 and US20070162989. Illustrative histochemical staining assays further include histochemical staining for neuronal specific markers, such as NgR, βIII tubulin, activin A, activin receptor Ia, activin receptor IIa, activin receptor Ib, activin receptor IIb, axin 1, chondroitin sulfate 4, chondroitin sulfate E, 2′,3′-Cyclic Nucleotide 3′-Phosphodiesterase (CNPase), Calbindin-D, Caspr2, Jagged 1, MAP2, Microtubule-Associated Proteins (MAPs), Myelin Basic Protein (MBP), NOTCH1, NOTCH2, NOTCH3, Nestin, Netrin-G1a, NeuroD1, NeuroD2, Neurofilament 200, Neurogenin 2, Neuronal Specific Enolase, Noggin, Receptor for Advanced Glycation Endproducts (RAGE), Reelin, Synaptophysin (SYP), Semaphorin 3A, Semaphorin 6A, Striatin, Survivin, Synapsin I, Synapsin II, Vimentin, Glutamic Acid Decarboxylase 67 (GAD67), N-Cadherin, Neural Cell Adhesion Marker (NCAM), Tau, Neuropilin-1, Neuropilin-2, Peripherin, NG2, cerebroglycan, glycipan, Neurocan, Oligodendrocyte Marker O1, Oligodendrocyte Marker O4, and Neurofibromin.

Alternatively, inhibition of CS mediated signaling such as CS-A mediated signaling, CS-C mediated signaling, or CS-E mediated signaling can be determined by (1) boundary crossing assays provided herein and as further described in M. Kaneko, T. Kubo, K. Hata, A. Yamaguchi, T. Yamashita, Neurosci. Lett. 423, 62 (2007); (2) growth cone collapse assays exemplified herein and as further described in U.S. Pat. No. 6,103,232; and (3) neurite outgrowth assays exemplified herein and as further described in R. J. Gilbert et al., Mol. Cell. Neurosci. 29, 545 (2005), R. Sivasankaran et al., Nat. Neurosci. 7, 261 (2004), M. Kaneko, T. Kubo, K. Hata, A. Yamaguchi, T. Yamashita, Neurosci. Lett. 423, 62 (2007), P. P. Monnier, A. Siena, J. M. Schwab, S. Henke-Fahle, B. K. Mueller, Mol. Cell. Neurosci. 22, 319 (2003), U.S. Patent Application Publication No. US20090104166, and in U.S. Pat. No. 6,207,639.

Additional procedures for assessing inhibition of CS mediated signaling including signaling via CS-A, CS-C, and/or CS-E mediated include assays for the inhibition of downstream mediators of CS mediated signaling. Exemplary methods include kinase assays for determining, e.g., MEK or EGFR kinase activity, immunoblots for detecting the phosphorylation level of downstream mediators in the CS sigaling pathway. Such downstream mediators include but are not limited to MEK, EGFR or RhoA western or dot blots. In some cases, calcium influx assays (see, e.g. Science, Vol 259, Issue 5091, 80-83) or the measurement of GTPase activity of RhoA can also be performed (see, e.g., J. Biomol Screen. 2009 February; 14(2):161-72. Epub 2009 Feb. 4, and US Patent Application Publication No. US20060115870) to assess inhibition of CS mediated signaling. In addition, a wide variety of assays are available to assess protein or mRNA expression levels of these signaling molecules. Non-limiting examples include microarray assays, northern blot assays, SAGE assays, digital PCR assays, ELISA assays, pull down assays, radio-immuno-precipitation assays, histochemical staining, and western blot assays.

Inhibition of CS mediated signaling may further be evidenced by inhibition of binding of one or more of CS-A, CS-C, or CS-E to cell surface or extracellular receptors. Exemplary methods include pull down assays such as biotin pull down assays, ELISA, surface plasmon resonance assays, fluorescence activated cell sorting assays, and fluorescence resonance energy transfer assays. These CS binding assays may be performed on intact cells expressing one or more of p75^{NTR}, NgR, Ephrin receptor A1, Ephrin receptor A2, Ephrin receptor A3, Ephrin receptor A4, Ephrin receptor A5, Ephrin receptor A6, Ephrin receptor A7, Ephrin receptor A8 or Ephrin receptor B3. Alternatively, these binding assays may be performed on purified protein or in cell or cell membrane extracts. These binding assays may further be performed in the presence of suspected modulators, antagonists, enhancers or agonists of the CS:receptor interaction. Theses modulators may include ephrins, ligands that activate Ephrin receptors, including but not limited to, Ephrin A1, Ephrin A2, Ephrin A3, Ephrin A4, Ephrin A5, Ephrin B1, Ephrin B2 and Ephrin B3.

The methods of the present invention further provide for administering one or more of the compositions provided herein in a physiological acceptable carrier. In some embodiments, the compositions of the present invention may be administered as a pharmaceutical composition comprising a physiologically acceptable carrier and an agent that inhibits CS mediated signaling (e.g. CS-E mediated signaling), mitigates CS-mediated neuronal growth inhibition, or promotes neuronal regeneration or neurite outgrowth. In some cases, the agent that mitigates CS-mediated neuronal growth inhibition is a CS polysaccharide binding agent, such as an anti-CS antibody. In some cases, the CS polysaccharide binding agent selectively binds one or more of CS-A, CS-C, or CS-E. In some cases, the CS polysaccharide binding agent selectively binds CS-E, such as an anti-CS-E antibody. The pharmaceutical composition may comprise any of the binding agents provided herein such as anti-CS antibodies including anti-CS-E antibodies. The compositions provided herein may further be administered in a physiologically acceptable carrier such as any of the carrier formulations provided herein, or any other physiological acceptable carrier known in the art for administering macromolecular or small molecule binding agents of the present invention.

Therapeutic formulations of the binding agent of the present invention are prepared for storage by mixing the binding agent having the desired degree of purity with optional physiologically acceptable-carriers, excipients or stabilizers (Remington's Pharmaceutical Sciences 16^{th} edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween, Pluronics™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

The formulations to be used for in vivo administration must be sterile. This is readily accomplished by filtration through sterile filtration membranes. Sustained-release preparations may be prepared. Suitable examples of sustained-release preparations include semipermeable matrices of solid hydrophobic polymers containing the binding agent of the present invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat No. 3,773,919), copolymers of L-glutamic acid and γ-ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated binding agents remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The methods provided herein further provide for administering a pharmaceutical composition of the present invention to a subject. The pharmaceutical compositions of the present invention may be administered by any suitable means, including parenteral, subcutaneous, intraperitoneal, intrapulmonary, intrathecal, and intranasal, and intralesional administration. Parenteral infusions include intramuscular, intrathecal, intravenous, intraarterial, intraperitoneal, or subcutaneous administration. In addition, the binding agents of the present invention may suitably be administered by pulse infusion, particularly with declining doses of the binding agent of the present invention. In some cases, the dosing is given by injections, such as intravenous, intrathecal, or subcutaneous injections, depending in part on whether the administration is brief or chronic. In other cases, the dosing is provided by a pump such as a mini-osmotic pump for long term delivery of the pharmaceutical composition, see e.g. U.S. Pat. Nos. 3,995,631, 4,320,758, and 7,335,193.

The binding agents and pharmaceutical compositions of the invention are particularly useful for parenteral administration, i.e., subcutaneously, intrathecally, intramuscularly or intravenously. The compositions for parenteral administration will commonly comprise a solution of a binding agent such as an antibody or fragment thereof of the invention or a cocktail thereof dissolved in an acceptable carrier, preferably an aqueous carrier. A variety of aqueous carriers may be employed, e.g., water, buffered water, 0.4% saline, 0.3% glycine, and the like. These solutions are sterile and generally free of particulate matter. These solutions may be sterilized by conventional, well-known sterilization techniques. The compositions may contain pharmaceutically acceptable auxiliary substances as required to approximate physiological conditions such as pH adjusting and buffering agents, etc. The concentration of the antibody or fragment thereof of the invention in such pharmaceutical formulation can vary widely, i.e., from less than about 0.5%, usually at or at least about 1% to as much as 15 or 20% by weight, and will be selected primarily based on fluid volumes, viscosities, etc., according to the particular mode of administration selected.

A pharmaceutical composition of the invention for intramuscular injection can be prepared to contain about 1 ml sterile buffered water, and about 50 mg of an antibody or fragment thereof of the invention. Similarly, a pharmaceutical composition of the invention for intravenous infusion could be made up to contain about 250 ml of sterile Ringer's solution, and 150 mg of a binding agent such as an antibody or fragment thereof of the invention. Actual methods for preparing parenterally administrable compositions are well known or will be apparent to those skilled in the art, and are described in more detail in, for example, Remington's Pharmaceutical Science, 15^{th} ed., Mack Publishing Company, Easton, Pa., hereby incorporated by reference herein.

The pharmaceutical compositions of the invention can be lyophilized for storage and reconstituted in a suitable carrier prior to use. This technique has been shown to be effective with many protein, peptide, small molecule, and nucleic acid based binding agents and art-known lyophilization and reconstitution techniques can be employed.

Depending on the intended result, the pharmaceutical composition of the invention can be administered for prophylactic and/or therapeutic treatments. In therapeutic application, compositions are administered to a patient already suffering from a disease, in an amount sufficient to cure or at least partially arrest the disease and its complications. In prophylactic applications, compositions containing the present binding agents or a cocktail thereof are administered to a patient not already in a disease state to enhance the patient's resistance.

Single or multiple administrations of the pharmaceutical compositions can be carried out with dose levels and patterns being selected by the treating physician. In any event, the pharmaceutical composition of the invention should provide a quantity of the binding agents (or fragments thereof) of the invention sufficient to effectively treat the patient.

For the prevention or treatment of disease, the appropriate dosage of the one or more binding agents of the present invention will depend on the type of disease to be treated, the severity and course of the disease, whether the binding agent of the present invention is administered for preventive or therapeutic purposes, previous therapy, the patient clinical history and response to the binding agent of the present invention, and the discretion of the attending physician. The binding agent of the present invention may be suitably administered to the patient at one time or over a series of treatments.

Depending on the type and severity of the neural injury or disease, about 1 μg/kg to 15 mg/kg (e.g., 0.1-20 mg/kg) of the binding agent of the present invention is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired neural growth occurs. However, other dosage regimens may be useful. The progress of this therapy is easily monitored by conventional techniques and assays. Dosages may begin to be administered within 1, 2, 3, 4, 5, 6, 7 or more days post injury.

The pharmaceutical composition comprising an inhibitor of CS mediated signaling such as a binding agent of the present invention may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the binding agent to be administered may be governed by such considerations, and is the minimum amount necessary to promote neural growth either in vivo or in vitro. The binding agent need not be, but may optionally be formulated with one or more agents currently used to prevent or treat the disorder in question. The effective amount of such other agents depends on the amount of the binding agent present in the formulation, the type of disorder or treatment, and other factors discussed above. These are generally used in the same dosages and with administration routes as used hereinbefore or about from 1 to 99% of the heretofore employed dosages.

In some embodiments, the pharmaceutical composition, binding agent and/or host cell of the present invention may be used to prevent or treat a neural injury or a neurodegenerative disease in a mammal. The mammal selected from the group consisting of a human, non-human primate, rodent, dog, cat, horse, cow, pig, sheep, rabbit, guinea pig, or goat. Subjects in need thereof may present, suffer from, be suspected of suffering from, or be susceptible to any one of a number of possible neural injuries or neurodegenerative diseases such as any of the injuries, diseases or conditions provided herein including but not limited to optic nerve injury, spinal cord injury, motor nerve injury, cholinergic neuron injury, blunt trauma, severing of a nerve, nerve fiber, or nerve tissue, or injuries due to environmentally, biologically (e.g. viral infection), physically, or chemically (e.g. nerve agent, or neurotoxin) induced trauma. Subjects in need thereof may present, suffer from, be suspected of suffering from, or be susceptible to any one of a number of possible neural injuries or neurodegenerative diseases such as any of the injuries, diseases or conditions provided herein including but not limited to Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjögren-Batten disease), Bovine spongiform encephalopathy (BSE), Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, Toxic encephalopathy, or any combination thereof.

The methods of the present invention further provide for contacting one or more of the pharmaceutical compositions of the present invention with one or more of extracellular matrix components or neuronal cells. Extracellular matrix components that may be contacted with a pharmaceutical composition of the present invention according to the methods of the present invention include but are not limited to proteoglycans such as CSPGs (e.g. CS-A, CS-C, CS-E), heparan sulfate, and keratan sulfate; non-proteoglycan polysaccharides such as hyaluronic acid; protein fibers such as collagen and elastin; cell surface proteins such as fibronectin and laminin; and enzymes such as urokinase plasminogen activator and matrix metalloproteinases. Neuronal cells that may be contacted with a pharmaceutical composition of the present invention according to the methods of the present invention include but are not limited to dopaminergic neurons, cortical neurons, cholinergic neurons, GABAergic neurons, glutamatergic neurons, dopaminergic neurons, serotonergic neurons, peripheral neurons, autonomic neurons, motor neurons, sensory neurons, spinal cord neurons, interneurons, unipolar neurons, bipolar neurons, multipolar neurons, Basket cells, Betz cells, Medium spiny neurons, Purkinje cells, Pyramidal cells, Renshaw cells, Granule cells, and anterior horn cells.

In one aspect, the present invention provides a method of producing a CS binding agent such as an agent that selectively binds one or more of CS-A, CS-C, or CS-E such as anti-CS-A, anti-CS-C, or anti-CS-E antibody, comprising culturing a host cell containing an isolated polynucleotide encoding an antibody of the present invention under conditions suitable for expression of the subject antibody. In some embodiments, the present invention encompasses a culture medium comprising a host cell disclosed herein. In other embodiments, the present invention further includes a culture fermentor comprising a plurality of host cells of the invention in a culture medium.

Fermentation is a standard process well known in the relevant art for the breakdown and re-assembly of biochemicals and biological molecules including proteins for industry, often in aerobic growth conditions. A fermentor or a bioreactor as used herein refers to an apparatus that maintains optimal conditions for the growth of microorganisms, e.g. host cells of the present invention, used in large-scale or small-scale fermentation and in the commercial production of biological macromolecules including but not limited to proteins, for example, antibodies, NgR, p75^{NTR}, Ephrin B3 receptor, Nogo, OMgp, or fragments thereof. Fermentation chambers may be used to produce these antibodies on a large scale. In some embodiments, the binding agent of the present invention can be produced in a culture fermentor.

### III. Compositions

The present invention provides one or more compositions for promoting neuronal growth or regeneration in a subject. In some embodiments, the compositions promote neuronal growth or regeneration by inhibiting CS-mediated signaling. For example, these compositions may comprise binding agents that selectively bind to CS proteoglycans or to CS proteoglycan receptors, or compositions that selectively inhibit signaling events downstream of CS proteoglycan binding to cell surface or extracellular proteins. In some embodiments, the present invention provides binding agents that selectively bind to one or more of CS-A, CS-C, or CS-E. In some embodiments, binding agents that selectively bind to one or more of CS-A, CS-C, or CS-E reduce or eliminate the inhibitory effect of said CS proteoglycans on neuronal growth or regeneration. For example, the binding agents may sequester these proteoglycans from binding to an endogenous receptor, thereby preventing the generation of an inhibitory signal in a neuron. In some cases, the binding agents that selectively bind to CS-E may reduce or eliminate the inhibitory effect of said CS proteoglycans on neuronal growth or regeneration.

Selective binding agents are agents that bind to a substrate, receptor or antigen with an affinity, or avidity for that substrate, receptor or antigen that is greater than for another substrate, receptor, or antigen. In some embodiments a binding agent that selectively binds CS exhibit an affinity, or avidity to CS that is greater than the affinity, or avidity of that agent for a glycan that is not CS such as for example heparan sulfate or for a molecule that is not a proteglycan such as a protein or peptide. In some embodiments, a binding agent of the present invention selectively bind one or more of CS-A, CS-C, or CS-E. Such binding agents that selectively bind one or more of CS-A, CS-C, or CS-E, may exhibit a greater affinity or avidity for one or more of CS-A, CS-C, or CS-E than that agent exhibits for other CS molecules, or than that agent exhibits for non-CS molecules or a combination thereof. For example, a binding agent of the present invention may selectively bind CS-A and exhibit reduced or undetectable binding to CS-C or CS-E or a combination thereof. Alternatively, a binding agent of the present invention may selectively bind CS-C and exhibit reduced or undetectable binding to CS-A or CS-E or a combination thereof. In still other cases, a binding agent of the present invention may selectively bind CS-E and exhibit reduced or undetectable binding to CS-A or CS-C or a combination thereof. In yet other embodiments, a binding agent of the present invention may selectively bind CS-E (for example) and exhibit reduced or undetectable binding to other, non CS, molecules. In some embodiments, selective binding to one or more of CS-A, CS-C, or CS-E may be assayed by the use of a carbohydrate array as provided herein. Other selective binding agents of the present invention include binding agents that selectively bind to CS receptors such as CS-E receptors including but not limited to NgR or p75^{NTR}.

In some embodiments, selective binding agents are provided that bind to one or more of CS-A, CS-C, or CS-E (e.g. CS-E) or a combination thereof with a high affinity. For example, selective binding agents are provided that bind CS-E with an affinity (e.g. Kd) greater than about 100 nM, 75 nM, 50 nM, 40 nM, 30 nM, 20 nM, 10 nM, 5 nM, 4 nM, 3 nM, 2 nM, or greater than about 1 nM. By “greater than” is meant with a stronger affinity. By way of example an affinity (e.g. Kd) of 10 nM may be considered greater than an affinity of 50 nM. In some cases, selective binding agents are provided that bind CS (e.g. CS-E) with an affinity between about 1 μM and about 1000 nM, between about 1 μM and about 750 nM, 1 μM and about 500 nM, 1 μM and about 250 nM, 1 μM and about 100 nM, 1 μM and about 50 nM, 1 μM and about 25 nM, 1 μM and about 10 nM, 10 μM and about 10 nM, 20 μM and about 10 nM, 50 μM and about 10 nM, 50 μM and about 5 nM, or about 1 μM, 2 μM, 3 μM, 5 μM, 10 μM, 20 μM, 40 μM, 50 μM, 100 μM, 150 μM, 200 μM, 300 μM, 500 μM, 750 μM, 1 nM, 2 nM, 4 nM, 4.3 nM, 5 nM, 10 nM, 20 nM, 30 nM, 40 nM, 50 nM, 100 nM, 200 nM. 300 nM, 400 nM, 500 nM, 750 nM or 1000 nM.

In other embodiments, selective binding agents are provided that bind CS-E and inhibit the binding of CS-E to other targets, ligands or receptors such as Nogo, Nogo receptor (NgR), p75^{NTR}, OMgp, or any fragment or soluble domain or derivative therefrom. For example, selective binding agents are provided that bind CS-E and inhibit binding to other targets, ligands or receptors with an inhibitor concentration (e.g. IC₅₀) of less than 1000 nM, less than 750 nM, less than 500 nM, less than 400 nM, less than 300 nM, less than 200 nM, less than 150 nM, less than 100 nM, less than 50 nM, less than 40 nM, less than 30 nM, less than 20 nM, or less than 10, 5, 4, 3, 2, or 1 nM. IC₅₀ values can be assessed by directly measuring binding of CS-E to other targets, ligands, or receptors as provided herein, or by measuring down stream signaling events provided by CS-E as provided herein.

Exemplary binding agents include but are not limited to proteins such as antibodies, Nogo, Nogo receptor (NgR), p75^{NTR}, OMgp, or any fragment or soluble domain or derivative therefrom which exhibits selective binding to one or more CS proteoglycans, such as for example one or more of CS-A, CS-C, or CS-E. Exemplary binding agents further include peptides such as for example the Nogo-66 antagonist peptide NEP 1-40. Exemplary binding agents further include aptamers such as polynucleic acids (DNA/RNA) or derivatives thereof including but not limited to pegylated or methylated derivatives which exhibit selective binding to one or more CS proteoglycans. Exemplary binding agents further include small molecules such as carbohydrate mimics that may bind to CS receptors such as for example P75^{NTR}, or NgR without triggering neuron growth inhibitory downstream signaling events. Exemplary binding agents still further include annexin domains (see e.g. U.S. Patent Application Publication No. 20030133939), and avimers (see e.g. Silverman et al., Nat. Biotechnol. (2005), 23: 1556-1561). Additionally, exemplary binding agents include agents containing carbohydrates, lipids, amino acids, small organic or inorganic molecules, or combinations thereof.

Antibody binding agents of the present invention include but are not limited to full length mammalian or avian antibodies such as IgG, IgA, IgM, IgD, IgY, or IgE molecules which consist of one or more heavy chains and one or more light chains. The heavy and light chains are comprised of constant and variable regions. The variable regions contain complementarity determining regions (CDRs), which along with the surrounding framework regions provide the antigen binding regions of the antibodies. Antibodies may further include full length camel or shark antibodies which contain a heavy chain, but lack a light chain. Antibodies of the present invention may be polyclonal or monoclonal. In some cases, antibodies may be a mixture of two or more different monoclonal antibodies. Antibodies provided herein also include humanized antibodies in which one or more immunogenic portions of the antibody have been removed, or removed and replaced with human or human like sequences, or in which one or more immunogenic portions of the antibody have been masked or derivatized, such as by pegylation or glycosylation. Antibodies provided herein also include antibody fusion proteins in which the antibody or a portion thereof is fused covalently or non-covalently with a peptide. Antibody fusion proteins may provide enhanced detection, enhanced efficacy, or enhanced antibody-mediated cytotoxicity. The antibodies may also be conjugated with drugs, toxins or therapeutic radioisotopes. Bispecific antibodies including hybrid antibodies which bind to more than one antigen are also included. The subject antibodies encompass naked antibodies, chimeric antibodies, conjugated antibodies and antibody fragments, which may be monospecific or multispecific.

Antibodies may further include fragments or full length antibodies of the isolated antibody 2D11-2A10 produced by a cell line deposited under ATCC accession number PTA10049 on May 5, 2009 and described in U.S. Pat. No. 7,745,584. Full length antibodies may comprise an isolated antibody comprising a heavy chain and a light chain, wherein said heavy chain comprises the three heavy chain complementarity determining regions (CDRs) of antibody 2D11-2A10 that is produced by a cell line deposited under ATCC accession number PTA10049, and wherein said light chain comprises the three light chain CDRs of said antibody 2D11-2A10, wherein the isolated antibody binds to a mammalian chondroitin sulfate E oligosaccharide comprising one or more disaccharides. Full length antibodies or fragments may also comprise one, two, or all three of the heavy and/or light chain CDRs of antibody 2D11-2A10. Theses antibodies can be further modified to humanized antibodies with improved immunogenicity profile or properly according to methods known in the art and disclosed herein.

Antibodies may further include fragments of full length antibodies such as described in U.S. Pat. No. 6,833,441 including single chain antibodies, single chain Fv's, disulfide stabilized single chain Fv's, and coiled coil stabilized single chain Fv's. In some cases, single chain Fv's may comprise heavy and light chain variable regions. In other cases, single chain Fv's may comprise just a heavy chain variable region, such as a camel or a shark single chain Fv, or just a light chain variable region. Antibodies may further include Fab fragments which comprise a portion of the heavy chain and the light chain. Antibodies may further include minibodies such as described in U.S. Pat. No. 5,837,821, or affibodies such as described in U.S. Pat. No. 5,831,012.

The antibodies of the present invention may be monoclonal which refers to an antibody from a population of substantially homogeneous antibodies, e.g., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target (e.g., CS-E), wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones.

It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations which typically include different antibodies directed against different determinants (e.g., epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol.340(5):1073-1093 (2004); Fellouse, Proc. Nat. Acad. Sci. USA 101(34):12467-12472 (2004); and Lee et al. J. Immunol. Methods 284(1-2):119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); U.S. Pat. No. 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995)).

Monoclonal antibodies herein include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

A “chimeric polypeptide” or a “chimeric polynucleotide” as used herein refers to an artificially constructed protein or polynucleotide comprising heterologous amino acid sequences or heterologous nucleic acid sequences, respectively. Chimeric proteins are proteins created through the joining of two or more genes, or portions of two or more genes, which originally coded for separate proteins. Translation of this fusion gene results in a single polypeptide with function properties derived from each of the original proteins. Such chimeric proteins are created artificially by recombinant DNA technology well known in the art.

The chimeric protein of the present invention is encoded by a heterologous nucleic acid sequence. As used herein, nucleic acid sequence is used interchangeably with polynucleotide, nucleotide, nucleotide sequence, nucleic acid and oligonucleotide. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, siRNA, shRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The heterologous sequence for production of a subject binding agent may comprise a vector, which is a nucleic acid molecule, preferably self-replicating, which transfers an inserted nucleic acid molecule into and/or between host cells. Vectors may include those that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An expression vector is a polynucleotide which, when introduced into an appropriate host cell, can be transcribed and translated into a polypeptide(s). In some embodiments, an expression vector comprises a polynucleotide encoding a chimeric anti-CS-E antibody. The encoded antibody can be modified, for example, disulfide bond formation, glycosylation, lipidation, acetylation, phosphorylation, or any other manipulation, such as conjugation with a labeling component. Furthermore, amino acid refers to either natural and/or unnatural or synthetic amino acids, including but not limited to glycine and both the D or L optical isomers, and amino acid analogs and peptidomimetics. Standard single or three letter codes are used to designate amino acids.

The polynucleotide sequence encoding a binding agent such as an antibody of the present invention can be expressed by a single or multiple vectors. The nucleic acid sequences can be arranged in any order in a single operon, or in separate operons that are placed in one or multiple vectors. Where desired, two or more expression vectors can be employed, each of which contains one or more heterologous sequences operably linked in a single operon. Linked refers to the joining together of two more chemical elements or components, by whatever means including chemical conjugation or recombinant means. Operably-linked refers to a juxtaposition wherein the components so described are in a relationship permitting them to function in their intended manner. For instance, a promoter sequence is linked, or operably linked, to a coding sequence if the promoter sequence promotes transcription of the coding sequence.

While the choice of single or multiple vectors and the use of single or multiple promoters may depend on the size of the heterologous sequences and the capacity of the vectors, it will largely dependent on the overall yield of a given glycoprotein that the vector is able to provide when expressed in a selected host cell. In some instances, two-operon expression system provides a higher yield of glycoproteins. The subject vectors can stay replicable episomally, or as an integral part of the host cell genome.

The heterologous sequence encoding a binding agent such as an antibody of the present disclosure can be under the control of a single regulatory element. In some cases, the heterologous nucleic acid sequences are regulated by a single promoter. In other cases, the heterologous nucleic acid sequences are placed within a single operon. In still other cases, the heterologous nucleic acid sequences are placed within a single reading frame.

Preparation of the subject nucleic acids can be carried out by a variety of routine recombinant techniques and synthetic procedures. Standard recombinant DNA and molecular cloning techniques are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. By Greene Publishing Assoc. and Wiley-Interscience (1987). Briefly, the subject nucleic acids can be prepared genomic DNA fragments, cDNAs, and RNAs, all of which can be extracted directly from a cell or recombinantly produced by various amplification processes including but not limited to PCR and rt-PCR.

Regulatory elements include, for example, promoters and operators, which can also be engineered to increase the expression of one or more heterologous sequences encoding a glycoprotein. A promoter is a sequence of nucleotides that initiates and controls the transcription of a nucleic acid sequence by an RNA polymerase enzyme. An operator is a sequence of nucleotides adjacent to the promoter that functions to control transcription of the desired nucleic acid sequence. The operator contains a protein-binding domain where a specific repressor protein can bind. In the absence of a suitable repressor protein, transcription initiates through the promoter. In the presence of a suitable repressor protein, the repressor protein binds to the operator and thereby inhibits transcription from the promoter.

In some embodiments of the present disclosure, promoters used in expression vectors are inducible. In other embodiments, the promoters used in expression vectors are constitutive. In some embodiments, one or more nucleic acid sequences are operably linked to an inducible promoter, and one or more other nucleic acid sequences are operably linked to a constitutive promoter. Non-limiting examples of suitable promoters for use in eukaryotic host cells include, but are not limited to, a CMV immediate early promoter, an HSV thymidine kinase promoter, an early or late SV40 promoter, LTRs from retroviruses, and a mouse metallothionein-I promoter.

The genes in the expression vector typically will also encode a ribosome binding site to direct translation (that is, synthesis) of any encoded mRNA gene product. Other regulatory elements that may be used in an expression vector include transcription enhancer elements and transcription terminators. See, for example, Bitter et al., Methods in Enzymology, 153:516-544 (1987).

An expression vector may be suitable for use in particular types of host cells and not others. One of ordinary skill in the art, however, can readily determine through routine experimentation whether a particular expression vector is suited for a given host cell. For example, the expression vector can be introduced into the host organism, which is then monitored for viability and expression of any genes contained in the vector.

The expression vector may also contain one or more selectable marker genes that, upon expression, confer one or more phenotypic traits useful for selecting or otherwise identifying host cells that carry the expression vector. Non-limiting examples of suitable selectable markers for eukaryotic cells include dihydrofolate reductase and neomycin resistance.

The subject vectors can be introduced into a host cell stably or transiently by variety of established techniques. For example, one method involves a calcium chloride treatment wherein the expression vector is introduced via a calcium precipitate. Other salts, for example calcium phosphate, may also be used following a similar procedure. In addition, electroporation (that is, the application of current to increase the permeability of cells to nucleic acids) may be used. Other transformation methods include microinjection, DEAE dextran mediated transformation, and heat shock in the presence of lithium acetate. Lipid complexes, liposomes, and dendrimers may also be employed to transfect the host cells.

Upon introduction of the heterologous sequence into a host cell, a variety of methods can be practiced to identify the host cells into which the subject vectors have been introduced. One exemplary selection method involves subculturing individual cells to form individual colonies, followed by testing for expression of the desired protein prodcut. Another method entails selecting host cells containing the heterologous sequence based upon phenotypic traits conferred through the expression of selectable marker genes contained within the expression vector. Those of ordinary skill can identify genetically modified host cells using these or other methods available in the art.

For example, the introduction of various heterologous sequences of the disclosure into a host cell can be confirmed by methods such as PCR, Southern blot or Northern blot hybridization. For example, nucleic acids can be prepared from the resultant host cells, and the specific sequences of interest can be amplified by PCR using primers specific for the sequences of interest. The amplified product is subjected to agarose gel electrophoresis, polyacrylamide gel electrophoresis or capillary electrophoresis, followed by staining with ethidium bromide, SYBR Green solution or the like, or detection of DNA with a UV detection. Alternatively, nucleic acid probes specific for the sequences of interest can be employed in a hybridization reaction. The expression of a specific gene sequence can be ascertained by detecting the corresponding mRNA via reveres-transcription coupled PCR, Northern blot hybridization, or by immunoassays using antibodies reactive with the encoded gene product. Exemplary immunoassays include but are not limited to ELISA, radioimmunoassays, and sandwich immunoassays.

Furthermore, the introduction of various heterologous sequences of the disclosure into a host cell can be confirmed by the enzymatic activity of an enzyme that the heterologous sequence encodes. The enzyme can be assayed by a variety of methods known in the art. In general, the enzymatic activity can be ascertained by the formation of the product or conversion of a substrate of an enzymatic reaction that is under investigation. The reaction can take place in vitro or in vivo.

The subject binding agents such as an antibody of the present invention can be produced by any methods known in the art. For instance, the subject antibody can be produced heterologously by expressing the antibody in a host cell. Suitable host cells can be used to make hybridomas for producing the subject antibodies of the present invention. A mouse or other appropriate host animal, (such as a hamster, goat, sheep, dog, horse, pig, rat, rabbit, dog, cat, or gerbil, amongst others) can be immunized as to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In one embodiment, myeloma cells are used that fuse efficiently, support stable high-level production of antibody by the selected antibody-producing cells, and are sensitive to a medium such as HAT medium. In some embodiments the myeloma cell lines are murine myeloma lines, (including, but not limited to, MOPC-21, MPC-11, SP-2 or X63-Ag8-653 cells), human myeloma cell lines (including, but not limited to, Karpas 707H, RPMI 8226, 8226 AR/NIP4-1, KM-2R, or U-266), or rat myeloma cell lines (including, but not limited to, YB2/3.0.Ag.20, YB2/0, Y3- Ag1.2.3, IR983F).

Culture medium in which hybridoma cells are growing is assayed for production of monoclonal antibodies directed against the antigen. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis. (see for example, Munson et al., Anal. Biochem. 107:220 (1980)).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones can then be suitably separated from the culture medium, ascites fluid, or serum by conventional antibody purification procedures such as, for example, protein A-Sepharose, hydroxylapatite chromatography, gel electrophoresis, dialysis, or affinity chromatography.

In some embodiments, a monoclonal antibody can be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain.

Conventional recombinant techniques can be employed to generate polypeptides encoding the heavy and light chains of a subject anti-CS antibody. For example, messenger RNA (mRNA) coding for heavy or light chain is isolated from a suitable source, such as mature B cells or a hybridoma culture, is obtained by employing standard techniques of RNA isolation purification and optionally size based isolation. cDNAs corresponding to mRNAs coding for heavy or light chain are then produced and isolated using techniques known in the art, such as cDNA library construction, phage library construction and screening or RT-PCR using specific relevant primers. In some embodiments, the cDNA sequence may be one that is wholly or partially manufactured using known in vitro DNA manipulation techniques to produce a specific desired cDNA. The cDNA sequence can then be positioned in a vector which contains a promoter in reading frame with the gene and compatible with the low-modified host cell. Numerous plasmids that contain appropriate promoters, control sequences, ribosome binding sites, and transcription termination sites, and optionally convenient markers are known in the art, these include but are not limited to, vectors described in U.S. Pat. Nos. 4,663,283 and 4,456,748. In one embodiment, the cDNA coding for the light chain and that coding for the heavy chain may be inserted into separate expression plasmids. In an alternative embodiment, the cDNA coding for the light chain and that coding for the heavy chain may be inserted together in the same plasmid, so long as each is under suitable promoter and translation control.

The expression vectors constructed above can then be used to transform a host cell. In some embodiments, the host cell is a wildtype host cell. In other embodiments, the host cell is modified, for example, contains one or more mutations. In one embodiment, the light and heavy chains may be transformed into separate modified host cell cultures, either of the same or of differing species. In an alternative embodiment, separate plasmids for light and heavy chain may be used to co-transform a single modified host cell culture. In another embodiment, a single expression plasmid containing both genes and capable of expressing the genes for both light and heavy chain may be transformed into a single modified host cell culture.

When heavy and light chains are coexpressed in the same host, the isolation procedure is designed so as to recover reconstituted antibody. This can be accomplished by conventional antibody purification procedures such as, for example, protein A-Sepharose, hydroxylapatite chromatography, gel electrophoresis, dialysis, or affinity chromatography.

In some embodiments, a monoclonal antibody purified by recombinant methods can be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain.

As noted above, a subject binding agent such as an antibody can be a humanized antibody, such as a human chimeric antibody, or a human complementary determining region (CDR) grafted antibody. Humanized forms of non-human (e.g., mouse, rat, hamster, goat, sheep, horse, cattle or rabbit) antibodies are chimeric antibodies that contain minimal sequence derived from non-human immunoglobulin. Humanized antibodies are typically human immunoglobulins (recipient antibody) in which residues from a hypervariable region of the recipient are replaced by residues from a hypervariable region of a non-human species (donor antibody) such as mouse, rat, goat, sheep, horse, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody comprises substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al, Nature 332:323-329 (1988); and Presta, Curr. Op. Struct. Biol. 2:593-596 (1992).

The term variable refers to certain portions of the variable domains differing extensively in sequence among antibodies and are used in the binding and specificity of each particular antibody for its particular antigen. However, the variability is typically not evenly distributed throughout the variable domains of antibodies. It is usually concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5^{th} Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term hypervariable region when used herein refers to the amino acid residues of an antibody which are responsible for antigen-binding. The hypervariable region generally comprises amino acid residues from a CDR (e.g residues 24-34 (L1), 50-56 (L2) and 89-97 (L3) in the light chain variable domain and 31-35 (HI), 50-65 (H2) and 95-102 (H3) in the heavy chain variable domain; Kabat et al, Sequences of Proteins of Immunological Interest, 5^{th} Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

A human chimeric antibody is an antibody which comprises an antibody heavy chain variable region (hereinafter referred to as “HV” or “VH”, the heavy chain being “H chain”) and an antibody light chain variable region (hereinafter referred to as “LV” or “VL”, the light chain being “L chain”), both of an animal other than human, a human antibody heavy chain constant region (hereinafter also referred to as “CH”) and a human antibody light chain constant region (hereinafter also referred to as “CL”). As the animal other than human, any animal such as mouse, rat, hamster, rabbit or the like can be used, so long as a hybridoma can be prepared there from.

A human chimeric antibody can be produced by obtaining cDNA's encoding heavy chain variable region (VH) and light chain variable region (VL) from a monoclonal antibody-producing hybridoma, inserting them into an expression vector for a host cell having genes encoding human antibody CH and human antibody CL to thereby construct a human chimeric antibody expression vector, and then introducing the vector into a low-fucosylation cell to express the antibody.

In regards to the heavy chain constant region (CH) of a human chimeric antibody, any CH can be used, so long as it is in the human immunoglobulin (hereinafter referred to as “hIg”) class. In some embodiments, the CH belongs to the hIgG class or one of the subclasses belonging to the hIgG class, such as hIgG1, hIgG2, hIgG3 and hIgG4. Likewise, in regards to the light chain constant region (CL) of human chimeric antibody, any CL can be used, so long as it belongs to the hIg class. In some embodiments, the light chain constant region (CL) of human chimeric antibody, belongs to the keppa class or lambda class.

A human CDR-grafted antibody can be produced by constructing cDNA's encoding variable regions in which CDR's of VH and VL of an antibody derived from an animal other than human are grafted into CDR's of VH and VL of a human antibody, inserting them into an expression vector for host cell having genes encoding human antibody CH and human antibody CL to thereby construct a human CDR-grafted antibody expression vector, and then introducing the expression vector into a modified host cell of the present disclosure to express the human CDR-grafted antibody.

Preferably the antibody of the present invention essentially retains the ability to bind antigen compared to the parental antibody. In some embodiments, the antibody of the present invention exhibits higher binding affinity to an antigen, for example, at least 1.1, 1.2, 1.3. 1.4, 1.5, 2, 3, 4, or 5 fold higher than a parental antibody. In other embodiments, the antibody of the present invention exhibits lower binding affinity to an antigen, for example, no less than 5%, 10%, 20%, 30%, 40%, or 50% of the binding affinity of a parental antibody to the antigen. The binding capability of the antibody of the present invention may be determined using techniques such as fluorescence activated cell sorting (FACS) analysis or radioimmunoprecipitation (RIA), for example.

In some embodiments, the anti-CS antibody of the present invention encompasses any form of CS binding unit other than a full-length antibody, such as an anti-CS binding unit other than a full length antibody that selectively binds one or more of CS-A, CS-C, or CS-E. A vector encoding a recombinant protein (such as a plasmid, or virus) using techniques standard in the art can be introduced into a host cell and the recombinant antibody may be any antibody fragments, such as Fv, Fab, scFV or diabody fragments.

The Fv fragment is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′).sub.2 antibody fragments originally were produced as pairs of Fab′ fragments which have hinge cysteines between them. Other chemical couplings of antibody fragments are also known.

Single-chain Fv (scFv) antibody fragments comprise the VH and VL domains of antibody, wherein these domains are present in a single polypeptide chain. In some embodiments the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994). Antibody scFv fragments are described in WO93/16185; U.S. Pat. No. 5,571,894; and U.S. Pat. No. 5,587,458, which are hereby incorporated by reference in their entirety, and may be produced by the modified host cells of the present disclosure.

The term diabodies refers to small antibody fragments with two antigen-binding sites, which fragments comprise a VH domain connected to a VL domain in the same polypeptide chain (VH-VL). By using a linker that is too short to allow pairing between the two domains on the same chain, the domains are forced to pair with the complementary domains of another chain and create two antigen-binding sites. Diabodies are described more fully in, EP 404,097; WO 93/11161; and Hollinger et al, Proc. Natl. Acad. Sci USA, 90:6444-6448 (1993), which are hereby incorporated by reference in their entirety. In some embodiments, the anti-CS antibody provided herein such as an anti-CS-E antibody or fragment thereof is an inhibitory antibody. Inhibitory antibodies may inhibit one or more biological activities of the antigen to which the antibody binds. For example, an inhibitory antibody can downregulate signal transduction of the corresponding antigen by inhibiting the activity of the antigen or inhibit expression of the antigen. In some embodiments, the antibody is a neutralizing antibody. A neutralizing antibody reduces or abolishes some biological activity of a soluble antigen or of a living microorganism, such as an infectious agent. Neutralizing antibodies may compete with the natural ligand or receptor for its antigen.

### Host Cells

An anti-CS antibody of the present disclosure, such as an anti-CS antibody that selectively binds to one or more of CS-A, CS-C, or CS-E can be produced by a host cell. A host cell includes an individual cell, cell culture, or cell line. Host cells include progeny of a single host cell. A host cell can be transfected with a heterologous sequence of the present disclosure. Host cells may be prokaryotic or eukaryotic, such as bacterial cells, fungal cells, animal cells, insect cells, plant cells and the like. Host cells are preferably capable of glycosylation.

Examples of bacterial host cells include microorganisms belonging to the genus Escherichia, Serratia, Bacillus, Brevibacterium, Corynebacterium, Microbacterium, Pseudomonas and the like. For example, bacterial host cells may include, but not be limited to, Escherichia coli XL1-Blue, XL2-Blue, DH1, MC1000, KY3276, W1485, JM109, HB101, No. 49, W3110, NY49, G1698, or TB1. Other bacterial host cells may include, but not be limited to, Serratia ficaria, Serratia fonticola, Serratia liquefaciens, Serratia marcescens, Bacillus subtilis, Bacillus amyloliquefaciens, Brevibacterium ammoniagenes, Brevibacterium immariophilum ATCC 14068, Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 13869, Corynebacterium acetoacidophilum ATCC 13870, Microbacterium ammoniaphilum ATCC 15354, Pseudomonas putida, Pseudomonas sp. D-0110 and the like.

Yeast host cells may include microorganisms belonging to the genus Saccharomyces, Schizosaccharomyces, Kluyveromyces, Trichosporon, Schwanniomyces, Pichia, Candida and the like, such as Saccharomyces cerevisiae, Schizosaccharomyces pombe, Kluyveromyces lactis, Trichosporon pullulans, Schwanniomyces alluvius, Candida utilis and the like.

Other examples of eukaryotic cells include animal cells such as mammalian cells. For example, host cells preferably include, but are not limited to, Chinese hamster ovary cells (CHO) or monkey cells, such as COS cells. The CHO cells may include, but not be limited to, CHO/dhfr⁻ or CHO/DG44 cells. The Chinese hamster ovary tissue-derived CHO cell includes any cell which is a cell line established from an ovary tissue of Chinese hamster (Cricetulus griseus). Examples include CHO cells described in documents such as Journal of Experimental Medicine, 108, 945 (1958); Proc. Natl. Acad. Sci. USA, 60 , 1275 (1968); Genetics, 55, 513 (1968); Chromosoma, 41, 129 (1973); Methods in Cell Science, 18, 115 (1996); Radiation Research, 148, 260 (1997); Proc. Natl. Acad. Sci. USA, 77, 4216 (1980); Proc. Natl. Acad. Sci., 60, 1275 (1968); Cell, 6, 121 (1975); Molecular Cell Genetics, Appendix I, II (pp. 883-900); and the like. In addition, CHO-K1 (ATCC CCL-61), DUXB11 (ATCC CCL-9096) and Pro-5 (ATCC CCL-1781) registered in ATCC (The American Type Culture Collection) and a commercially available CHO-S (Life Technologies, Cat # 11619) or sub-cell lines obtained by adapting the cell lines using various media can also be exemplified.

In an alternative embodiment the parent cell line is derived from a lymphocytic lineage cell line, such as a B cell line. The host cell may be from cell lines used in hybridoma production. They can be myeloma cells, such as from murine myeloma lines, such as, but not limited to, MOPC-21, MPC-11, NSO, SP-2, Sp2/0, S194, and X63-Ag8-653 cells; human myeloma cell lines , such as, but not limited to, Namalwa, Karpas 707H, RPMI 8226, 8226 AR/NIP4-1, KM-2R, and U-266; or rat myeloma cell lines, such as, but not limited to, YB2/0, YB2/3.0.Ag.20, Y3- Ag1.2.3, IR983F. Cell lines, such as HeLa, HEK-293, NIH3T3, COS, CHO, NSO, PER.C6, K562, L1.2, JY, BHK, K562, 293F, 3T3, and Jurkat may also be used in the present disclosure.

Examples of insect host cells include Spodoptera frugiperda ovary cells, such as Sf9 and Sf21 (Baculovirus Expression Vectors, A Laboratory Manual, W. H. Freeman and Company, New York (1992)); a Trichoplusia ni ovary cell such as High 5 (manufactured by Invitrogen); and the like. Examples of plant host cells include plant cells of tobacco, potato, tomato, carrot, soybean, rape, alfalfa, rice, wheat, barley and the like.

In some embodiments, the host cell is a non-lymphocytic cell. A lymphocyte is a type of white blood cell in the vertebrate immune system. Lymphocytes typically include T cells, B cells and natural killer (NK) cells. A non-lymphocytic cell encompasses any type of cell that is not a lymphocyte. The host cell of the invention may have a species origin selected from the group consisting of human, mouse, rat, fruit fly, worm, yeast and bacterium. The host cell may be derived from a suitable tissue including but not limited to blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The host cells producing the subject antibodies may be derived from any developmental stage including embryo and adult stages, as well as developmental origin such as ecotodermal, mesodermal, and ectodermal origin. In some embodiments, the host cells are CHO, NS0, SP2/0, HEK293, PER.C6 or YB2/0 cells.

### IV. Examples

### Example 1

### Dorsal Root Ganglion (DRG) Neurite Outgrowth Assay

Neurite outgrowth assays using dorsal root ganglions are useful for assaying the effects of the methods and compositions of the present invention in promoting neuronal regeneration or neurite outgrowth or in reducing or eliminating CS-E mediated signaling such as for example CS-E mediated inhibition of neuronal growth. Acid-treated, 15-mm round German glass coverslips (Carolina Biologicals) are coated with poly-DL-ornithine (P-Orn; Sigma-Aldrich) in pH 6.5-8.5 borate buffer (approximately 0.5 mg/ml) for about 2 hr at about 37 ° C. and approximately 5% CO₂. The coverslips are then washed with sterile water and dried in air. CSPGs (approximately 1 μg/ml; Chemicon), CSPGs treated with chondroitinase ABC (Seikagaku; 1-40 mU ChABC per pg CSPG incubated for about 2 hr at approximatley 37° C.; complete cleavage verified by SDS-PAGE), polysaccharides enriched in the CS-A, CS-C or CS-E sulfation motifs (approximately 1 μg/ml based on glucuronic acid content; Seikagaku super special grade, purified from sturgeon notochord, shark cartilage and squid cartilage, respectively), or synthetic glycopolymers (approximately 1 μg/ml) are added to the coated coverslips for about 2 hr at 37° C. and 5% CO₂. DRGs are dissected from day 4-10 chick embryos, incubated in 0.0125%-0.25% trypsin w/ EDTA for about 15 min at 37° C., triturated to dissociate to single cell suspensions, and grown on the coverslips coated with the above-mentioned substrata. Cells are grown in a growth medium composed of DMEM/F12, 10% horse serum, approximately 50 ng/ml NGF (Sigma-Aldrich), and Insulin-Transferrin-Selenium-X Supplement (Invitrogen) for about 12 hr. In the case of antibody blocking experiments, approximately 0.1 mg/ml of the anti-CS-E (1), anti-CS-A (2H6; Seikagaku), or IgG control antibody (Pierce) is added into the medium at time T=0 and co-incubated with cells for about 12 hr. Cells are fixed in paraformaldehyde (PFA) with 5-15% sucrose, immunostained using a rabbit anti-βIII tubulin antibody (Sigma) and subsequently an Alexa Fluor 488-conjugated goat anti-mouse IgG secondary antibody (Pierce), imaged using a Nikon TE2000-S fluorescent microscope equipped with Metamorph software, and quantified using the NIH software Image J. The data are statistically analyzed using the one-way ANOVA; n=50-200 cells per experiment. Under the conditions described herein, the results show that the methods and compositions of the present invention are effective at promoting neurite outgrowth.

### Example 2

### Growth Cone Collapse Assay

Growth cone collapse assays are useful for assaying the effects of the methods and compositions of the present invention in promoting neuronal regeneration or neurite outgrowth or in reducing or eliminating CS-E mediated signaling such as for example CS-E mediated inhibition of neuronal growth. 8-Well Lab-Tek® II CC2™ Slides (Electron Microscopy Sciences) are coated with P-Orn in pH 6.5-8.5 borate buffer for about 2 hr at 37° C. and 5% CO₂, then coated with laminin (approximately 10 μg/ml; Invitrogen) for about 2 hr at 37° C. and 5% CO₂. DRG explants are dissected from E6-10 chick embryos and grown on the P-Orn/laminin substratum for about 24 hr, treated with the indicated polysaccharides (at approximately 10 μg/ml) or glycopolymers (at approximately 10 μg/ml) for about 30 min, fixed in PFA with 5-15% sucrose, stained using rhodamine phalloidin (Pierce), and imaged using a Nikon TE2000-S fluorescent microscope. The percentage of collapsed growth cones is expressed as the percentage ratio of the number of collapsed growth cones over the number of total growth cones (including collapsed and uncollapsed). P-values are determined using one-way ANOVA; n=50-100 growth cones per experiment, using results from at least five independent experiments. Under the conditions described herein, the results show that the methods and compositions of the present invention are effective at preventing growth cone collapse.

### Example 3

### Boundary Crossing Assay

Boundary crossing assays are useful for assaying the effects of the methods and compositions of the present invention in promoting neuronal regeneration or neurite outgrowth or in reducing or eliminating CS-E mediated signaling such as for example CS-E mediated inhibition of neuronal growth. CS polysaccharides at various concentrations mixed with Texas Red (at approximately 0.5 mg/ml; Invitrogen) are spotted at the center of P-Orn-coated coverslips (about 5 μl) for about 2 hr at 37° C. and 5% CO₂. Cerebella are dissected from P5-9 Sprague Dawley rats, incubated in 0.0125%-0.25% trypsin w/ EDTA for about 15 min at 37° C., triturated to dissociate to single cell suspensions, purified on a discontinuous Percoll gradient, and cultured on the coated coverslips for about 48 hr. Cells are fixed in PFA with 5-15% sucrose, immunostained using an anti-β tubulin III antibody, imaged using a Nikon TE2000-S fluorescent microscope and Metamorph software, and quantified using the NIH software Image J. Axons growing toward the boundary and within 10 μm distance of the boundary are evaluated. The percentage of axons that cross the boundary over the total axons is quantified. P-values are determined using one-way ANOVA; n=30-50 axons per experiment. Under the conditions described herein, the results show that the methods and compositions of the present invention are effective at promoting an increase in the number of axons that cross the boundary.

### Example 4

### Cerebellar Granule Neuron (CGN) Neurite Outgrowth Assay

CGN neurite outgrowth assays are useful for assaying the effects of the methods and compositions of the present invention in promoting neuronal regeneration or neurite outgrowth or in reducing or eliminating CS-E mediated signaling such as for example CS-E mediated inhibition of neuronal growth. Nitric acid-treated, 15-mm round German glass coverslips are coated with P-Orn in pH 6-8.5 borate buffer (at approximately 50 μg/ml) for about 1 hr at 37° C. and 5% CO₂. CSPGs (at approximately 1 μg/ml) or polysaccharides enriched in the CS-A, CS-C, or CS-E sulfation motifs (at approximately 1 μg/ml) are coated on the P-Orn-coated coverslips overnight at 37° C. and 5% CO₂. Cerebella from P5-9 Sprague Dawley rats are dissected, purified as described above, and cultured on the coated coverslips at a cell density of about 200 cells/mm². Neurons are grown in DMEM/F12, 1% FBS, and N1 supplement for about 24 hr.

In one example, inhibitors against MEK (PD98059, 25 μM; Calbiochem), EGFR (AG1478, 15 nM; Calbiochem), ROCK (Y27632, 5 μM; Calbiochem) and JNK (JNK Inhibitor II, 10 μM; Calbiochem) are added in solution at the start of culturing, and neurons are grown for 24 hr. For neurite outgrowth experiments from p75 knockout mice, cerebella from P5-7 mice, either C57BL/6 wild-type or p75−/− knockout mice, are dissected and purified as described above. Neurons are plated at a density of 200-300 cells/mm2 and cultured for about 24 hr. Cells are then fixed in 4% PFA/10% sucrose, immunostained with neuronal specific anti-class III β-tubulin antibody (TUJ1; Covance), and evaluated for neurite outgrowth. Neurons are imaged using a Nikon TE2000-S fluorescent microscope and quantified using the NIH software Image J. Statistical analysis is performed using the one-way ANOVA; n=50-200 cells per experiment. Under the conditions described herein, the results show that the methods and compositions of the present invention are effective at promoting neurite outgrowth.

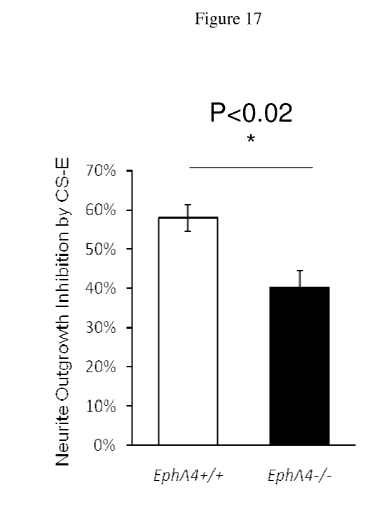

One of the proteins shown herein to specifically bind CS-E-enriched polysaccharides is the Eph receptor A4 (EphA4), suggesting that EphA4 is involved in CS-E signaling. In one example, CGN neurons from EphA4-deficient mice were examined for effects on neurite outgrowth. Mice were dissected and purified as described above. Neurons were plated, cultured, and fixed as described above and immunostained an anti-bIII tubulin antibody. Neurons were imaged using a Nikon TE2000-S fluorescent microscope and quantified using the NIH software Image J. Statistical analysis is performed using the one-way ANOVA; n=50-200 cells per experiment; p value <0.02. Results show that CGN neurons from EphA4-deficient mice exhibit reduced inhibition of neurite outgrowth by CS-E as compared to control mice (FIG. 17). Under the conditions described herein, the results show that the methods and compositions of the present invention are effective at promoting neurite outgrowth.

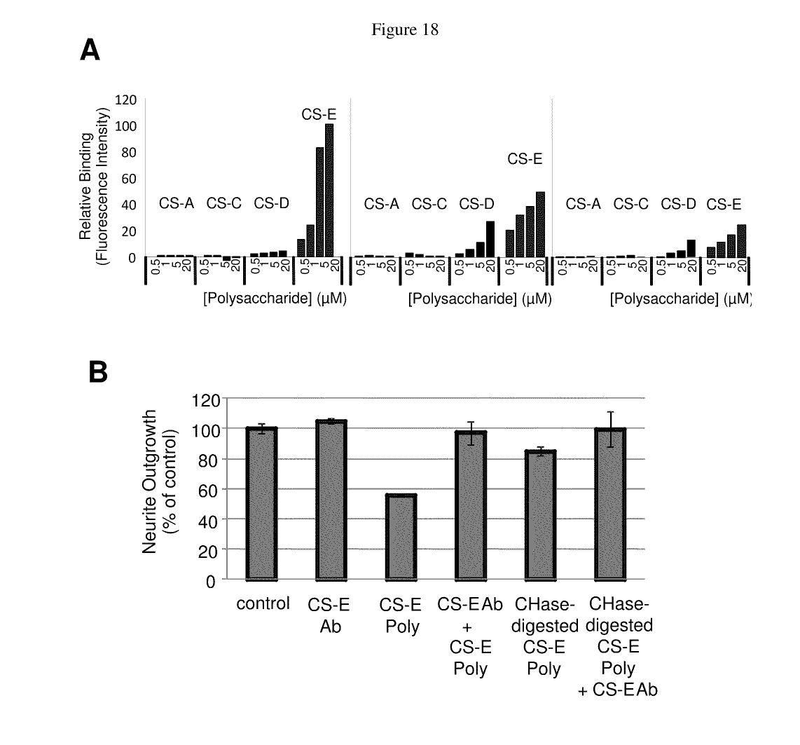

In another example, the results show that CS-E antibody blocks the inhibitory effects of CS-E polysaccharides on rat cerebellar granule neurons (FIG. 18B). P7 rat CGNs are grown for 24 hours on CS-E (1 μg/ml) in the presence of the CS-E antibody (CS-E Ab; 10 μg/ml). The CS-E antibody completely blocks the inhibition of neurite outgrowth caused by CS-E and CSPGs. Digestion of CS-E polysaccharides with chondroitinaseABC (CHase) results in a small extent of residual inhibition, which is completely blocked by the CS-E antibody. These results suggest that CS-E protein interactions play an important role in the CS-E signaling pathway and selective intervention of this pathway is able to block the inhibitory effects of CS-E polysaccharides and promote neurite outgrowth. Under the conditions described herein, the results show that the methods and compositions of the present invention are effective at promoting neurite outgrowth.

### Example 5

### Carbohydrate Microarray Assays

Carbohydrate microarray assays are useful for screening for or identifying binding agents that selectively bind one or more carbohydrates including but not limited to polysaccharides such as proteoglycans including CSPGs such as CS-A, CS-C, or CS-E. Carbohydrate microarrays are generated by spotting approximately 1 nL of heparin (Sigma), hyaluronic acid (Sigma), dermatan sulfate (Sigma), heparan sulfate (Neoparin), chondroitin (Seikagaku), chondroitin sulfate (Seikagaku), or keratan sulfate (Seikagaku) polysaccharide solutions onto poly-L-lysine-coated slides using a Microgrid II arrayer (Biorobotics; Cambridge, UK) at room temperature and 50% humidity. The concentrations of the solutions range from 250 nM to 40 μM, and are calibrated to one another using the carbazole assay for uronic acid residue. A given concentration of each polysaccharide is spotted five to ten times at different positions on the array. A boundary is created around the polysaccharide spots on the slides using a hydrophobic slide marker (Super Pap Pen, Research Products International) and the slides are blocked with 5-10% fetal bovine serum (FBS) in PBS with gentle rocking at 37 ° C. for about 1 hr, followed by a brief rinse with PBS. Human NgR-Fc or mouse EphB2-Fc (R & D Systems) is reconstituted in 1% BSA in PBS, added to the bound region on the slides in 100 μl quantities at a concentration of 1-2 μM, and incubated at room temperature for about 3 hr. The slides are briefly rinsed three times with PBS, and then incubated with a goat anti-human IgG antibody conjugated to Cy3 (1:5000 in PBS) for 1 hr in the dark with gentle rocking. After rinsing two times with PBS and once with H₂O, the microarray is analyzed at 532 nm using a GenePix 5000a scanner, and fluorescence quantification is performed using GenePix 6.0. Binding of the anti-CS-E antibody is evaluated using 100 μl of a 1 μg/ml (˜7 nM) solution of antibody and a goat anti-mouse IgG secondary antibody conjugated to Cy3 (1:5000 in PBS). Under the conditions described herein, the results show that the compositions of the present invention are selective binders of one or more of CS-A, CS-C, or CS-E.

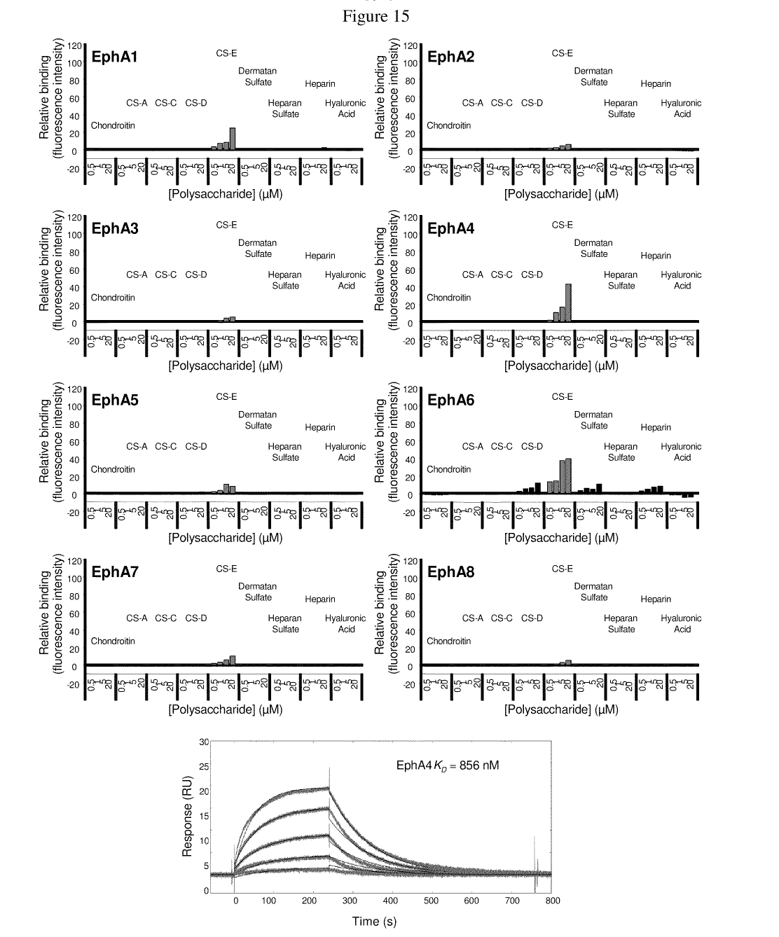

In one example, Ephrin receptor family proteins were screened for binding to CS-E-enriched polysaccharides. The binding of proteins EphA1, EphA2, EphA3, EphA4, EphA5, EphA6, EphA7 and EphA8 were examined relative to binding to Chondroitin, CS-A, CS-C, CS-D, CS-E, Dermatan Sulfate, Heparan Sulfate, Heparan, and Hyaluronic Acid at varying polysaccharide concentrations of 0.5, 1, 5, and 20 μM (FIG. 15). Results from this example demonstrate that EphA4 selectively binds to CS-E-enriched polysaccharides.

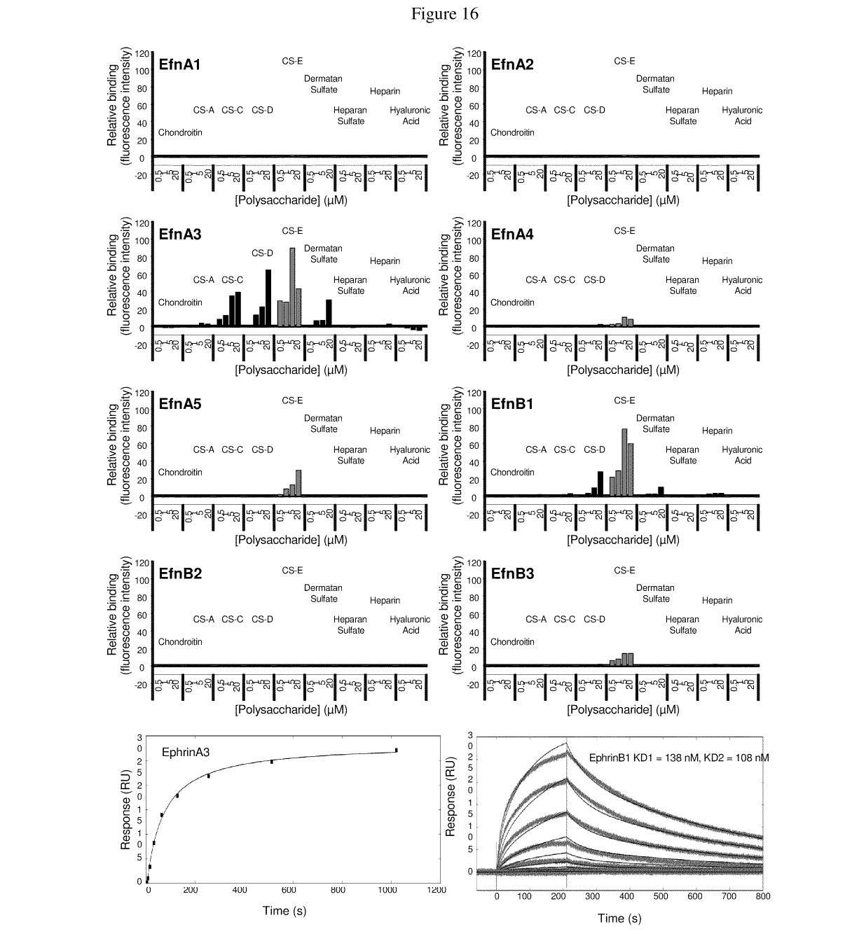

In another example, Ephrin family proteins, a large family of protein ligands that activate Eph receptors, were screened for binding to CS-E-enriched polysaccharides. The binding of proteins EfnA1, EfnA2, EfnA3, EfnA4, EfnA5, EfnB1, EfnB2, and EfnB3 were examined relative to binding to Chondroitin, CS-A, CS-C, CS-D, CS-E, Dermatan Sulfate, Heparan Sulfate, Heparan, and Hyaluronic Acid at varying polysaccharide concentrations of 0.5, 1, 5, and 20 μM (FIG. 16). Results from this example demonstrate that EfnA3 and EfnB1 selectively bind to CS-E-enriched polysaccharides.

In another example, competition assays were utilized in the context of the carbohydrate microarray assay. In the competition binding example described, these results demonstrate that CS-E antibody can compete for binding of Nogo Receptor (NgR) to CS-E polysaccharides on carbohydrate arrays (FIG. 18). In FIG. 18A, the CS-E antibody blocks binding of NgR-Fc to CS-E on the array. The binding reagents mixed are: NgR alone (200 nM, left), NgR and CSE- Ab (200 nM and 10 μM, respectively, middle) or CS-E Ab alone (10 μM, right). CS-E antibody competition for CS-E binding to NgR demonstrate specific binding of CS-E to NgR. In addition, it suggests the importance of CS-E interactions with NgR in the CS-E signaling cascade.

### Example 6

### Oligosaccharide Microarray Assays

Oligosaccharide microarray assays are useful for screening for or identifying binding agents that selectively bind one or more carbohydrates including but not limited to polysaccharides such as proteoglycans including CSPGs such as CS-A, CS-C, or CS-E. Immobilization of oligosaccharides may be performed by conjugation of oligosaccharides to 1,2-(bisaminooxy)ethane. Ozonolysis of the anomeric allyl group and linkage of CS compounds 1-41,2 to 1,2-(bisaminooxy)ethane3 proceeds as follows: oligosaccharide (0.51 μmol) is dissolved in MeOH (500 μL) and cooled to −78° C. O₃ is bubbled through the reaction until a blue color persists (1 min). The reaction is then purged with N₂ until colorless, quenched with Ph₃P beads (3 mg), and gradually warmed to rt over 12 h. The reaction is filtered and the product concentrated to afford the desired aldehyde as a white solid. The aldehyde (0.51 μmol) is then reacted for 14 h at rt with 1,2-(bisaminooxy)ethane hydrochloride (1.4 mg, 15 μmol) that has been dissolved in H₂O (100 μL) and pH adjusted to 5.0 with 1 M NaOH. The resulting oxime product is purified using a SepPak C18 column (500 mg, H₂O) and Sephadex G-10 (CS-E disaccharide, H₂O) or Sephadex G-25 (tetrasaccharides, H₂O) to afford a white solid in quantitative yield (0.51 μmol). CS-A aminooxy: ESI MS: m/z: calculated for C₃₂H₄₈N₄Na₃O₃₁S₂: 1117.1; found 1117.0. CS-C aminooxy: ESI MS: m/z: calculated for C₃₂H₄₈N₄Na₃O₃₁S₂: 1117.1; found 1117.0. CS-E aminooxy: ESI MS: m/z: calculated for C₃₂H₄₆N₄Na₅O₃₇S₄: 1321.0; found 1321.0. CS-E di amminooxy: ESI MS: m/z: calculated for C₁₈H₂₈N₃Na₂O₂₀S₂: 716.1; found 716.0. Mass spectra are obtained on a PerkinElmer/Sciex API 365 triple quadrupole/electrospray tandem mass spectrometer in the Protein/Peptide MicroAnalytical Laboratory at the California Institute of Technology.

The relative concentrations of the aminooxy oligosaccharides are calibrated to one another using the carbazole assay for uronic acid residues. Briefly, the acid borate reagent (1.5 mL of 0.80 g sodium tetraborate, 16.6 mL H2O, and 83.3 mL H2SO4) is added to 20-mL glass vials with Teflon caps. The aminooxy oligosaccharides (50 μL of a 0.2 mg/mL stock in H₂O) are added and the solution placed in a boiling H2O bath for 10 min. Following addition of the carbazole reagent (50 μL of 0.1% w/v carbazole in 100% EtOH), the solution is boiled for 15 min. The absorbance is read at 530 nm and compared to a D-glucuronolactone standard in H2O.

Solutions of the aminooxy oligosaccharides (in 300 mM NaH₂PO₄, pH 5.0, 10 μL/well in a 384-well plate) are arrayed on Hydrogel Aldehyde slides (NoAb Biodiscoveries) using a Microgrid II arrayer (Biorobotics) to deliver sub-nanoliter volumes at rt and 50% humidity. Concentrations of carbohydrates range from 0-500 μM. The resulting arrays are incubated in a 70% humidity chamber at rt for 12 h and then stored in a low humidity, dust-free dessicator. The pH and reaction time are optimized to provide maximum immobilization of the compound. Non-specific attachment of CS oligosaccharides lacking the aminooxy linker (e.g., compounds 1-4) is not observed. Prior to use, the arrays are outlined with a hydrophobic pen (Super Pap Pen, Research Products International) to create a boundary for the binding agent treatments and rinsed three times with H2O. The slides are then blocked by treatment with NaBH4 (125 mg) in 140 mM NaCl, 2.7 mM KCl, 5.4 mM Na₂HPO₄, and 1.8 mM KH₂PO₄ (phosphate buffered saline, PBS, 50 mL) at rt for 5 min with gentle rocking and washed five times for 3 min with PBS. For all incubations, the slides are placed in a covered pipette tip box. Human TNF-α (Peprotech), FGF-1 (R&D Systems; both reconstituted to 2 μM in 0.1% Triton X-100 in PBS), cell culture supernatant containing monoclonal anti-CS-A antibody, or cell culture supernatant containing monoclonal anti-CS-E antibody (both 1:1 in 0.1% Triton X-100 in PBS) are spotted onto the slides in 250 μt quantities, and incubated statically at rt for 2 h. The slides are then washed as previously described and incubated with the appropriate primary antibody [anti-TNF-α (Peprotech) or anti-FGF-1 (R&D Systems); 1:1000 in 0.1% Triton X-100 in PBS] for 2 h at rt with gentle rocking. Following the incubation, the slides are washed as previously described and treated in the dark at rt with a secondary IgG antibody conjugated to Cy3 (Amersham; 1:5000 in 0.1% Triton X-100 in PBS) at rt for 1 h with gentle rocking. The slides are washed three times for 2 min with PBS, two times for 1 min with H2O, and dried under a gentle stream of N₂. Microarrays are analyzed at 532 nm using a GenePix 5000a scanner, and fluorescence quantification is performed using GenePix 6.0 software after correction for local background. Each binding agent is analyzed in triplicate, and an average fluorescence intensity of at least five spots for a given carbohydrate concentration is observed. All solutions used for the carbohydrate microarrays are sterile-filtered through a 0.2 μm syringe filter prior to use. Under the conditions described herein, the results show that the compositions of the present invention are selective binders of one or more of CS-A, CS-C, or CS-E.

### Example 7

### ELISA Assay

ELISA assays can be used for detecting protein binding to a receptor or substrate, detecting the presence of a protein or carbohydrate, quantifying protein expression, or quantifying protein activity. ELISA assays can also be used for identifying agents that disrupt the binding between CS and a known binding partner. Antigens are absorbed on Nunc Maxisorp 384-well plates. For CSPG binding assays, CSPGs (at approximately 10 μg/ml; in about 25 μl) are incubated in each well for about 2 hr. For CS binding assays, streptavidin (at approximately 20 μg/ml; in about 50 μl) is absorbed in each well for about 1 hr, followed with biotinylated CS (at approximately 20 μg/ml; in about 50 μl) for about 1 hr. After blocking with 1-10% BSA in PBS, the anti-CS-E antibody (approximately 25 μl of 20 μg/ml or indicated concentrations in 1-3% BSA in PBS) is incubated in each well for about 2 hr. Following incubation with horseradish peroxidase-conjugated anti-mouse IgG secondary antibody, the plate is developed with TMB substrate (3,3′,5,5′-tetramethylbenzidine; Pierce) for about 20 min and quenched with 1-3M H₂SO₄. The absorption at 450 nm is recorded on a PerkinElmer Victor plate reader. Experiments are repeated in triplicate, and data representing the average values (±SD, error bars) are reported for one representative experiment. Under the conditions described herein, the results show that the methods and compositions of the present invention are effective at inhibiting CS-E mediated signaling.

### Example 8

### Pull-Down Assays with Biotinylated CS

Pull down assays can be useful for identifying proteins which bind to CS (e.g. CS-A, CS-C, or CS-E). Pull down assays can also be used for identifying agents that disrupt the binding between CS and a known binding partner. Full-length human p75 cDNA (amino acids 1-427; Open Biosystems) is ligated into a pcDNA3 vector (Invitrogen), modified to fuse an HA tag to the 5′ end of the insert. Human NgR (amino acids 27-473; imaGenes) is ligated into a modified pAPtag-5 vector (GenHunter) which fuses a secretion signal sequence and a c-myc tag to the 3′ end of the insert. COS-7 cells are transfected with either NgR-myc, p75-HA, or both using the Lipofectamine (Invitrogen) method. Cells are lysed about 2 days after transfection with 0.5-2% Triton X-100 in PBS containing a protease inhibitor mixture (Roche) by rocking for 15-90 min at 4° C. The lysates are clarified by centrifugation at 5-25,000 rpm for 1-30 min.

Biotin is attached to the free amino group of the residual core peptides of CS-C and CS-E polysaccharides (Seikagaku). Briefly, CS-C and CS-E (about 2 mg each) are dissolved about 1 mL of 0.015-0.1 M NaHCO₃ for about 30 min at room temperature. EZ-Link Sulfo-NHS-LC-LC-Biotin (approximately 0.25 mg; Pierce) is dissolved in about 1 mL of H₂O and added to each CS sample. The solution is mixed at room temperature for about 1-10 hr, lyophilized, resuspended in H₂O, and subjected to gel filtration using Sephadex G-50 or G-25 (Amersham) to remove excess biotin.

Biotinylated CS-C or CS-E (approximately 280 μg each) in 100-500 μl of PBS is added to streptavidin agarose resin (about 140 μl; Pierce) and incubated at room temperature for about 1 hr. The supernatant is removed, and the resin washed 1-5 times with about 500 μl of PBS to remove unconjugated CS. Clarified COS-7 cell lysates are diluted 1:5 to 1:20 with PBS, and further diluted approximately 1:2 with H₂O. Diluted lysate (about 500 μl, corresponding to approximately 0.8 mg/ml total protein) is precleared by incubation with unconjugated streptavidin agarose resin (e.g. 30 μl) with mixing for about 1 hr at 4° C. The supernatant is collected, added to either CS-C or CS-E streptavidin agarose resin (e.g. 30 μl), and incubated with mixing for about 4 hr at 4 ° C. The supernatant is removed, and the resin is washed 1-5 times with about 500 μl PBS. Resin is boiled with about 30 μl of 2X loading dye (100 mM Tris, 200 mM DTT, 4% SDS, 0.10% bromophenol blue, 20% glycerol), and the eluates are collected and separated by SDS-PAGE. NgR-myc and p75-HA are detected by immunoblotting with myc-tag (Cell Signaling) or HA-tag (Sigma) antibodies. Under the conditions described herein, the results show that the methods and compositions of the present invention are effective at inhibiting the binding of CS-E to NgR, p75^{NTR}, or other binding partners of CS-E including but not limited to Nogo, OMgp, and Ephrin B3 receptor.

### Example 9

### Surface Plasmon Resonance

Surface plasmon resonance experiments can be used to provide quantitative measurements of the binding kinetics or affinity between two or more molecules such as proteins, peptides, and small molecules. All experiments are performed on a Biacore T100 at 25° C. using a Sensor Chip CM5 with a running buffer composed of 0.01 M HEPES, pH 7.4, 0.15 M NaCl, 3 mM EDTA, 0.05% Surfactant P20 (HBS-EP⁺). To analyze the binding of anti-CS-E antibody to the CS-E tetrasaccharide, both control and active flow cells are exposed to a 1:1 mixture of N-hydroxysuccinimide hydroxysuccinimide (NHS) and 1-ethyl-3,3-dimethylaminopropyl-carbodiimide (EDC) for 1-10 min at a flow rate of about 10 μL·min⁻¹. Next, 1-10 mM carbohydrazine is injected at the same flow rate for about 5-15 min. Ligand is covalently attached to the surface by injecting a 0.1-1 mM solution of synthetic CS-E tetrasaccharide bearing an aldehyde group on a reducing-end linker, onto the active flow cell briefly at a high flow rate (approximately 10 s, 60 μL·min⁻¹), followed by a 20 min injection of 0.1 M sodium cyanoborohydride in 0.1 M sodium acetate pH 4.0 at about 2 μL·min⁻¹. Because of the low molecular weight of the CS-E tetrasaccharide, it may be difficult to observe the amount of ligand bound to the surface. Instead, approximately 500 nM of anti-CS-E antibody is injected into both the control and active flow cell to test the response. The amount of ligand is increased accordingly until an adequate response is observed. The kinetics of the anti-CS-E antibody/CS-E tetrasaccharide interaction is determined by approximately 300 s long injections of anti-CS-E antibody at about 30 μL·min⁻¹. The dissociation is monitored for about 900 s before the surface is regenerated with a 30 s injection of 6 M guanidine HCl. The resulting sensorgrams are fit to the bivalent analyte model. Affinity analysis is measured by injecting the antibody for about 3600 s at 5 μL·min⁻¹. After 3600 s, the surface is regenerated with a 60 s injection at about 10 μL·min⁻¹. The data is analyzed by plotting the response at equilibrium versus concentration and fitting the resulting curve to the Langmuir equation.

For the NgR interaction with CS-E-enriched polysaccharides, all flow cells are activated with NHS and EDC following the manufacturer's amine coupling protocol. Streptavidin (at approximately 1 μM, 0.01 M NaOAc, pH 5.0) is conjugated to the activated surfaces until saturation, followed by ethanolamine blocking. Biotinylated CS-E, composed of ˜61.5% of the CS-E disaccharide motif, the remainder of which is a mixture of CS-A, CS-C, and unsulfated disaccharides, is immobilized to flow cell 2 to give an R_{L} of 25 response units (RU); biotinylated CS-C is immobilized to flow cell 4 to give a similar R_{L}. Flow cells 1 and 3 are used as controls to subtract bulk response. NgR-Fc is passed over the surface at 25° C. with a flow rate of about 80 μL·min⁻¹ for 240 s, and the dissociation monitored for 600 s. The surface is regenerated by three injections of 90 s of 2.5 M MgCl₂ at 30 μL·min⁻¹. NgR-Fc injection results in binding of NgR-Fc to CS-E polysaccharides with two apparent kinetic processes, presumably caused by variations in the density of CS-E motifs along the polysaccharide chain. No binding is detected to CS-C-enriched polysaccharides. The resulting sensorgrams are analyzed using Biacore T100 evaluation software V2.0 and fit to the heterogeneous ligand model with the value of bulk refractive index (RI) set to zero. Under the conditions described herein, the results show that the methods and compositions of the present invention are effective at selectively binding to CS-E. The assay further shows that NgR and p75 bind to CS-E but not CS-C.

In another example, utilizing the surface plasmon resonance methods described, data was generated to quantify measurements of the binding kinetics between CS-E and the binding partner EphA4. The results show (FIG. 15, bottom) that EphA4 binds to CS-E-enriched polysaccharides with a dissociation constant (K_{d}) of 699 nM, demonstrating a strong binding affinity and suggesting that EphA4 may be an important target for CS-E-enriched polysaccharides.

In another example, utilizing the surface plasmon resonance methods described, data was generated to quantify measurements of the binding kinetics between CS-E and the binding partner EfnA3. The results show that EfnA3 binds to CS-E-enriched polysaccharides with a dissociation constant (K_{d}) of 65 nM, demonstrating a strong binding affinity and suggesting that EfnA3 may be an important target for CS-E-enriched polysaccharides.

In another example, utilizing the surface plasmon resonance methods described, data was generated to quantify measurements of the binding kinetics between CS-E and the binding partner EfnB1. The results show (FIG. 16, bottom) that EfnB1 binds to CS-E-enriched polysaccharides with a dissociation constant (K_{d}) of 138 nM, demonstrating a strong binding affinity and suggesting that EfnB1 may be an important target for CS-E-enriched polysaccharides.

### Example 10

### Immunostaining of Retinal and Optic Nerve Sections

Histochemical staining of retinal and optic nerve sections can be used to assess nerve growth, neurite outgrowth, growth cone collapse, neuron survival, and neuronal regeneration. At 1-14 days post injury, mice are given an overdose of pentobarbital and are transcardially perfused with 4% PFA. Eyeballs still attached with the optic nerve are dissected and post-fixed in 4% PFA overnight. Following cryoprotection with 30% sucrose in PBS, tissues are embedded in O.C.T. and serial sectioned for 5-20 μm along the longitudinal direction of the nerve. For immunofluorescence labeling, the sections are washed with PBS, pre-incubated in a blocking buffer (approximately 1% BSA, 0.3%TX-100 in PBS) and followed by sequential incubations with primary antibodies, including mouse anti-CS-E (1:200), goat-anti-CTB (1:4000), or rabbit anti-IβIII-tubulin antibodies (Invitrogen). The sections are then reacted with biotinylated anti-goat IgG (1:200) and visualized with Alexa Fluor 546-conjugated streptavidin (1:400) or with a Cy3-conjugated goat anti-mouse or -rabbit IgG. The retinal cryosections are mounted with Vectashield and visualized under a Nikon fluorescence microscope. To quantify the number of CTB-positive regenerating axons, the number of regenerating axons is counted at 125 μm stepwise from the crush site of the optic nerve. The total number of regenerating axons is estimated. To quantify the number of surviving retinal ganglion cells, the total number of βIII-tubulin positive cells is counted in at least 3 retinal sections per retina. Under the conditions described herein, the results show that the methods and compositions of the present invention are effective at promoting neurite outgrowth, nerve growth, neuron survival, and neuronal regeneration, and are also effective at inhibiting growth cone collapse.

### Example 11

### Optic Nerve Regeneration Assay

The optical nerve regeneration assay can be used to assess nerve growth, neurite outgrowth, growth cone collapse, neuron survival, and neuronal regeneration. Immediately after crush injury in the optic nerve of adult mice, gelfoam that has been soaked in a solution containing control IgG, anti-CS-E antibody (1.7 mg/ml), chondroitinase ABC (chABC; 50 U/ml), or chABC (50 U/ml) plus anti-CS-E (1.7 mg/ml) antibody is placed around the crush site of the nerve and replaced at day three and six after injury. In other groups of mice, mice that received an intravitreal injection of CPT-cAMP (100 mM, 2 μl) alone or CPT-cAMP plus anti-CS-E (1.7 mg/ml) antibody treatment placed around the crush site of the nerve are studied. To label retinal ganglion cell axons, 2 of a solution containing an anterograde axon tracer, CTB, is injected intravitreally 3 days before mice were sacrificed. The extent of axonal regrowth is assessed 2 weeks after injury. Under the conditions described herein, the results show that the methods and compositions of the present invention are effective at promoting neurite outgrowth, nerve growth, neuron survival, and neuronal regeneration, and are also effective at inhibiting growth cone collapse.

### Example 12

### Axon Regeneration and Motor Function Recovery Assay in Mice

The axon regeneration and motor function assay in mice can be used to assess the efficacy of the methods and compositions of the present invention in promoting the enhancement of locomotor function, bladder function, neuromuscular functional recover or walking in a subject.

Surgical methods: For surgery, mice are anesthetized with an IP injection of Avertin. Hair is removed by shaving, and the skin is treated with betadine. A midline incision is made over cervical spinous processes and paravertebral muscles are separated from the vertebrae. A laminectomy is performed at T8 or C5. Dorsal hemisections are performed using a Moria micro knife. After producing the spinal cord injuries, the muscle is sutured in layers, and the skin is closed with wound clips. Post-operatively, animals receive saline, dextrose, Baytril, and Buprenorphine subcutaneously and are placed on a warming blanket at 37° C. overnight. Bladders are manually expressed for the first week.

Assessing hindlimb motor function after T8 lesions: Hindlimb motor function during locomotion is assessed at 2, 7, 14, 21, 28, 35, and 42 days post-injury using the BBB and BMS scales. Kinematic analyses are carried out at 22 and 43 days; hindlimb placement on a horizontal ladder with variably spaced rungs is be assessed at 20 and 40 days post-injury.

Measuring forepaw function after C5 lesions using a grip strength meter: Forepaw grip strength is repeatedly measure over time in mice and rats using a Grip Strength Meter distributed by SciPro, Inc. The task takes advantage of the natural tendency of mice to reach out and grasp nearby objects. In brief, mice are handled and trained until they became accustomed to having one paw restrained with tape. The tape prevents that foot from being used to grasp the bar so that grip strength can be measured independently for each paw. For testing, mice are held by the base of the tail close to the bar of the grip strength meter, which they grasp with the free paw. The mice are pulled gently away until they release their grip, and the device measures the maximal force exerted before the paw releases its grip. This task offers considerable advantages because maximal grip strength is remarkably consistent from animal to animal and over prolonged periods of repeated testing.

Assessing corticospinal tract (CST) regeneration/sprouting: Regenerative growth of CST axons is assessed as described herein using histochemical staining methods. Under the conditions described herein, the methods and compositions of the present invention can be effective at promoting CST regeneration.

### Example 13

### CS Mediated Rho Activation Assay

The Rho activation assay can be used to assay for inhibitors of CS mediated Rho activation. Transfected COS-7 cells are treated with CS-E in combination with Nogo, OMgp or no protein, and RhoA-GTP levels are measured using methods known in the art (See e.g., Nature Neuroscience 7, 221-228 (2004); Neuron 45, 353-359 (2005); and Journal of Cell Biology 157, 565-570 (2002)). Briefly, the RhoA-binding domain of the effector protein Rhotekin can be used to affinity precipitate the GTP-bound form of RhoA. The amount of precipitated GTP-RhoA can then be determined for example by blotting or ELISA. RhoA activation levels are compared to those obtained using Nogo or OMgp alone. The results of these assays demonstrate that the methods and compositions of the present invention are effective at inhibition of CS mediated signaling.

### Example 14

### Cortical Legion Immunostaining Assay

Observing the increased expression of a protein in response to neural injury further emphasizes its importance in the tissue response signaling chain of events. In one example, expression of the CS-E motif was assessed after brain injury in vivo in rats as described herein using histochemical staining methods. Sections from rat brains with cortical lesions at 1 day, 3 days, and 7 days post lesion were immunostained with the CS-E antibody. Increased immunostaining around the lesion was observed at all three time points, and levels of staining appeared highest at 3 days post-lesion. At 1 day post lesion, there was a diffuse increase in immunostaining confined to the region immediately adjacent to the lesion. There was staining of cells throughout the brain and a few cells near the lesion exhibited higher levels of punctate staining. At 3 days post lesion, levels of staining around the lesion appeared higher, and the general cellular staining throughout the brain seemed a bit more intense. By 7 days post-lesion, the staining decreased at the lesion and throughout the brain.

Immunostaining was also assessed 2 days following a dorsal funiculus lesion of the rat spinal cord. As with cortical lesions, there was a general increase in immunostaining in the lesion margin, and some cells around the lesion expressed CS-E at a fairly high level. Some cells in the gray matter also stained for CS-E, but not as intensely as those near the lesion. Dorsal and ventral white matter also stained for CS-E in a pattern that resembled neurofilament expression.

Together, these data demonstrate that the CS-E sulfation motif is upregulated shortly after brain or spinal cord injury in vivo. These findings also suggest that the time course for treating with the CS-E antibody should likely be early, given the rapid increase in CS-E expression within 1-2 days post injury.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of promoting neuronal regeneration in a subject, comprising: administering to said subject in need thereof a pharmaceutical composition comprising a physiologically acceptable carrier and an agent that inhibits chondroitinsulfate-E (CS-E) signaling, wherein said agent is present in an amount effective in promoting neurite outgrowth in said subject.

2. The method of claim 1, wherein the agent is a chondroitin-sulfate-E (CS-E) binding agent.

3. A method of treating a subject suffering from a neural injury, comprising: administering to said subject in need thereof a pharmaceutical composition comprising a therapeutically effective amount of a chondroitin-sulfate-E (CS-E) binding agent and a physiologically acceptable carrier.

4. A method of reducing chondroitin sulfate proteoglycan (CSPG)-mediated inhibition of neuronal growth, comprising: contacting a biological sample comprising extracellular matrix components with a chondroitin-sulfate-E (CS-E) binding agent under conditions sufficient to reduce CS-E-signaling in said neuronal cell.

5. The method of any one of claims 2-4, wherein the chondroitin-sulfate-E (CS-E) binding agent is an antibody that binds selectively to CS-E.

6. The method of claim 5, wherein said antibody is a monoclonal antibody.

7. The method of any one of claims 2-4, wherein the chondroitin-sulfate-E (CS-E) binding agent is soluble protein that binds selectively to CS-E.

8. The method of claim 7, wherein the soluble protein comprises a domain of p75, NgR, Nogo, OMgp, or Eph receptor B3 that selectively binds to CS-E.

9. The method of claim 7, wherein the soluble protein comprises a domain of Ephrin A3, Eph receptor A4, or Ephrin B1 each of which selectively binds to CS-E.

10. The method of any one of claims 2-4, wherein the chondroitin-sulfate-E (CS-E) binding agent inhibits CS-E signaling as evidenced by inhibition of activation of RhoA in a neuronal cell or a cell expressing p75^{NTR}, or inhibition of binding of CS-E to a surface of a neuronal cell or a cell expressing p75^{NTR}.

11. The method of claim 10, wherein the chondroitin-sulfate-E (CS-E) binding agent inhibits CS-E-mediated neuronal growth cone collapse.

12. The method of claim 2, wherein the chondroitin-sulfate-E (CS-E) binding agent promotes neurite outgrowth in vitro.

13. The method of claim 2, wherein the chondroitin-sulfate-E (CS-E) binding agent exhibits a binding affinity as measured in Kd value of about 50 nM or less in an in vitro binding assay.

14. The method of claim 2, wherein said administration of chondroitin-sulfate-E (CS-E) binding agent results in an enhancement of locomotor function, grasping, bladder function, neuromuscular functional recovery, or walking in said subject.

15. The method of claim 2, wherein the chondroitin-sulfate-E (CS-E) binding agent is administered within 1 to 2 days post injury.

16. The method of claim 2, wherein the subject has suffered from an injury selected from the group consisting of spinal cord injury, optic nerve injury, motor nerve injury, stroke, and a combination thereof or a neurodegenerative disease.

17-29. (canceled)
